SEQUENCE LISTING (56 sequences)

SEQ ID NO: 1-2 - skipped (2 entries)

SEQ ID NO: 3 - protein, 18 aa, synthetic construct
  REGION 1..18: note = Rigid linker 1
EAAAAKEAAAAKEAAAAK

SEQ ID NO: 4 - protein, 613 aa, Pseudomonas aeruginosa
  REGION 1..613: note = Full length Pseudomonas Exotoxin A (PE)
AEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQTQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANADVVSLTCPVAAGECAGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 5 - protein, 26 aa, synthetic construct
  REGION 1..26: note = PE translocation peptide (PE280-305, minimal)
GWEQLEQCGYPVQRLVALYLAARLSW

SEQ ID NO: 6 - protein, 34 aa, synthetic construct
  REGION 1..34: note = PE translocation peptide (PE280-313)
GWEQLEQCGYPVQRLVALYLAARLSWNQVDQVIR

SEQ ID NO: 7 - protein, 46 aa, synthetic construct
  REGION 1..46: note = PE translocation peptide (PE268-313)
PLETFTRHRQPRGWEQLEQCGYPVQRLVALYLAARLSWNQVDQVIR

SEQ ID NO: 8 - protein, 61 aa, synthetic construct
  REGION 1..61: note = PE translocation peptide (PE253-313)
GGSLAALTAHQACHLPLETFTRHRQPRGWEQLEQCGYPVQRLVALYLAARLSWNQVDQVIR

SEQ ID NO: 9 - protein, 112 aa, synthetic construct
  REGION 1..112: note = PE translocation peptide (PE253-364)
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAN

SEQ ID NO: 10 - protein, 293 aa, Shigella dysenteriae
  REGION 1..293: note = Full length Shiga toxin (Stx) subunit A
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 11 - protein, 293 aa, Escherichia coli
  REGION 1..293: note = Full length Shiga-like toxin I (Slt-I) subunit A
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 12 - protein, 8 aa, synthetic construct
  REGION 1..8: note = Stx translocation peptide (Stx240-247, minimal)
LNCHHHAS

SEQ ID NO: 13 - protein, 12 aa, synthetic construct
  REGION 1..12: note = Stx translocation peptide (Stx240-251)
LNCHHHASRVAR

SEQ ID NO: 14 - protein, 37 aa, synthetic construct
  REGION 1..37: note = Stx translocation peptide (Stx211-247)
PDYHGQDSVRVGRISFGSINAILGSVALILNCHHHAS

SEQ ID NO: 15 - protein, 41 aa, synthetic construct
  REGION 1..41: note = Stx translocation peptide (Stx211-251)
PDYHGQDSVRVGRISFGSINAILGSVALILNCHHHASRVAR

SEQ ID NO: 16 - protein, 84 aa, synthetic construct
  REGION 1..84: note = Stx translocation peptide (Stx168-251)
ALRFRQIQRGFRTTLDDLSGRSYVMTAEDVDLTLNWGRLSSVLPDYHGQDSVRVGRISFGSINAILGSVALILNCHHHASRVAR

SEQ ID NO: 17 - protein, 261 aa, Homo sapiens
  REGION 1..261: note = Full length CD40 ligand (CD40L1-261)
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 18 - protein, 215 aa, synthetic construct
  REGION 1..215: note = Truncated CD40 ligand (CD40L47-261)
HRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 19 - protein, 154 aa, synthetic construct
  REGION 1..154: note = Truncated CD40 ligand (CD40L108-261, also referred to as 18sCD40L)
ENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 20 - protein, 246 aa, synthetic construct
  REGION 1..246: note = Anti-CD40 scFv (VH-L-VL)
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSSGGGGSGGGGSGGGGSQSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 21 - protein, 246 aa, synthetic construct
  REGION 1..246: note = Anti-CD40 scFv (VL-L-VH)
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLGGGGGSGGGGSGGGGSEVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 22 - protein, 119 aa, synthetic construct
  REGION 1..119: note = VH of the anti-CD40 scFv
EVQLLESGGGLVQPGGSLRLSCAASGFTFSTYGMHVWRQAPGKGLEWLSYISGGSSYIFYADSVRGRFTISRDNSENALYLQMNSLRAEDTAVYYCARILRGGSGMDLWGQGTLVTVSS

SEQ ID NO: 23 - protein, 112 aa, synthetic construct
  REGION 1..112: note = VL of the anti-CD40 scFv
QSVLTQPPSASGTPGQRVTISCTGSSSNIGAGYNVYWYQQLPGTAPKLLIYGNINRPSGVPDRFSGSKSGTSASLAISGLRSEDEADYYCAAWDKSISGLVFGGGTKLTVLG

SEQ ID NO: 24 - protein, 10 aa, synthetic construct
  REGION 1..10: note = VH CDR1
GFTFSTYGMH

SEQ ID NO: 25 - protein, 26 aa, synthetic construct
  REGION 1..26: note = VH CDR2
GKGLEWLSYISGGSSYIFYADSVRGR

SEQ ID NO: 26 - protein, 13 aa, synthetic construct
  REGION 1..13: note = VH CDR3
CARILRGGSGMDL

SEQ ID NO: 27 - protein, 15 aa, synthetic construct
  REGION 1..15: note = VL CDR1
CTGSSSNIGAGYNVY

SEQ ID NO: 28 - protein, 7 aa, synthetic construct
  REGION 1..7: note = VL CDR2
GNINRPS

SEQ ID NO: 29 - protein, 12 aa, synthetic construct
  REGION 1..12: note = VL CDR3
CAAWDKSISGLV

SEQ ID NO: 30 - protein, 4 aa, synthetic construct
  REGION 1..4: note = ER retention sequence
KDEL

SEQ ID NO: 31 - protein, 12 aa, synthetic construct
  REGION 1..12: note = ER retention sequence
KKDLRDELKDEL

SEQ ID NO: 32 - protein, 13 aa, synthetic construct
  REGION 1..13: note = ER retention sequence
KKDELRDELKDEL

SEQ ID NO: 33 - protein, 13 aa, synthetic construct
  REGION 1..13: note = ER retention sequence
KKDELRVELKDEL

SEQ ID NO: 34 - protein, 12 aa, synthetic construct
  REGION 1..12: note = ER retention sequence
KDELKDELKDEL

SEQ ID NO: 35 - protein, 28 aa, synthetic construct
  REGION 1..28: note = CD28 consensus sequence
  SITE 8: note = X is I or L
  SITE 10: note = X is V, F or A
  SITE 11: note = X is M or L
  SITE 17: note = X is L or I
TDIYFCKXEXXYPPPYXDNEKSNGTIIH

SEQ ID NO: 36 - protein, 28 aa, synthetic construct
  REGION 1..28: note = CD28-activating peptide (minimal)
TDIYFCKIEVMYPPPYLDNEKSNGTIIH

SEQ ID NO: 37 - protein, 53 aa, synthetic construct
  REGION 1..53: note = CD28-activating peptide
NCDGKLGNESVTFYLQNLYVNQTDIYFCKIEVMYPPPYLDNEKSNGTIIHVKG

SEQ ID NO: 38 - protein, 98 aa, Human papillomavirus type 16
  REGION 1..98: note = Antigen HPV16 E7 protein
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 39 - protein, 104 aa, Human papillomavirus type 18
  REGION 1..104: note = Antigen HPV18 E7 protein
HGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQ

SEQ ID NO: 40 - protein, 154 aa, Hepatitis B virus
  REGION 1..154: note = Antigen HBV X protein (HBx; full length)
MAARMCCQLDPARDVLCLRPVGAESRGRPLPGPLGALPPSSASAVPADHGSHLSLRGLPVCSFSSAGPCALRFTSARRMETTVNAPWSLPTVLHKRTIGLSGRSMTWIEEYIKDCVFKDWEELGEEIRLKVFVLGGCRHKLVCSPAPCNFFTSA

SEQ ID NO: 41 - protein, 108 aa, Hepatitis B virus
  REGION 1..108: note = Antigen HBV preS1 protein
MGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPI

SEQ ID NO: 42 - protein, 190 aa, Hepatitis C virus
  REGION 1..190: note = Antigen HCV core protein (full length)
MSTNPKPQRKTKRNTNRRPQDVKFPGGGQIVGGVYLLPRRGPRLGVRATRKTSERSQPRGRRQPIPKARRPEGRTWAQPGYPWPLYGNEGMGWAGWLLSPRGSRPNWGPTDPRRRSRNLGKVIDTLTCGFADLMGYIPLVGAPLGGVARALAHGVRVLEDGVNYATGNLPGCSFSIFLLALLSCLTTPAS

SEQ ID NO: 43 - protein, 1273 aa, Severe acute respiratory syndrome coronavirus 2 (SARS-CoV-2)
  REGION 1..1273: note = Antigen SARS-CoV2 spike protein
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 44 - protein, 187 aa, Homo sapiens
  REGION 1..187: note = Antigen SSX2
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDD

SEQ ID NO: 45 - protein, 314 aa, Homo sapiens
  REGION 1..314: note = Antigen MAGE-A3
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 46 - protein, 180 aa, Homo sapiens
  REGION 1..180: note = Antigen NY-ESO-1
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 47 - protein, 296 aa, Homo sapiens
  REGION 1..296: note = Antigen iLRP
FSGALDVLQMKEEDVLKFLAAGTHLGGTNLDFQMEQYIYKRKSDGIYIINLKRTWEKLLLAARAIVAIENPADVSVISSRNTGQRAVLKFAAATGATPIAGRFTPGTFTNQIQAAFREPRLLVVTDPRADHQPLTEASYVNLPTIALCNTDSPLRYVDIAIPCNNKGAAHSVGLMWWMLAREVLRMRGTISREHPWEVMPDLYFYRDPEEIEKEEQAAAEKAVTKEEFQGEWTAPAPEFTATQPEVADWSEGVQVPSVPIQQFPTEDWSAQPATEDWSAAPTAQATEWVGATTDWS

SEQ ID NO: 48 - protein, 279 aa, Homo sapiens
  REGION 1..279: note = Antigen WT12-281
GSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPI

SEQ ID NO: 49 - protein, 406 aa, Homo sapiens
  REGION 1..406: note = Antigen RNF43
SGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPAHPLICGPPGLDKRLLPETPGPCYSNSQPVWLCLTPRQPLEPHPPGEGPSEWSSDTAEGRPCPYPHCQVLSAQPGSEEELEELCEQAVSGGHQLQLAALWPWLLMATLQAGFGRTGLVLAAAVESERSAEQKAIIRVIPLKMDPTGKLNLTLEGVFAGVAEITPAEGKLMQSHPLYLCNASDDDNLEPGFISIVKLESPRRAPAHPLICGPPGLDKRLLPETPGPCYSNSQPVWLCLTPRQPLEPHPPGEGPSEWSSDTAEGRPCPYPHCQVLSAQPGSEEELEELCEQAV

SEQ ID NO: 50 - protein, 284 aa, Homo sapiens
  REGION 1..284: note = Antigen CEA-NE3
KLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDTPIISPPDSSYLSGANLNLSCHSASNPSPQYSWFVNGTFQQHTQVLLIAKIQPNNNGTYACFVSNLATGRNNSIVKSITV

SEQ ID NO: 51 - protein, 528 aa, synthetic construct
  REGION 1..528: note = Fusion protein CD40L47-261-TPE-E7
MHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAKDEL

SEQ ID NO: 52 - protein, 467 aa, synthetic construct
  REGION 1..467: note = Fusion protein 18sCD40L-TPE-E7
MENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLEFGSELEAAAAKEAAAAKEAAAAKRARYKRGWEQLEQCGYPVQRLVALYLAARLSWTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAKDEL

SEQ ID NO: 53 - protein, 535 aa, synthetic construct
  REGION 1..535: note = Fusion protein E7-TStx-CD40L47-261
MKLTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAPDYHGQDSVRVGRISFGSINAILGSVALILNCHHHASRVAREFGSELEAAAAKEAAAAKEAAAAKHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 54 - protein, 474 aa, synthetic construct
  REGION 1..474: note = Fusion protein E7-TStx-18sCD40L
MKLTMGSSHHHHHHSSGLVPRGSHMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPAEAAAAKEAAAAKEAAAAKEAAAAKAHGPKATLQDIVLHLEPQNEIPVDLLCHEQLSDSEEENDEIDGVNHQHLPARRAEPQRHTMLCMCCKCEARIKLVVESSADDLRAFQQLFLNTLSFVCPWCASQQTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 55 - protein, 541 aa, synthetic construct
  REGION 1..541: note = Fusion protein HBx-preS1-TStx-18sCD40L
MKLTMGSSHHHHHHSSGLVPRGSHMAARMCCQLDPARDVLCLRPVGAESRGRPLPGPLGALPPSSASAVPADHGSHLSLRGLPVCSFSSAGPCALRFTSARRMETTVNAPWSLPTVLHKRTIGLSGRSMTWIEEYIKDCVFKDWEELGEEIRLKVFVLGGCRHKLVCSPAPCNFFTSARGRAEAAAAKEAAAAKEAAAAKEAAAAKAEFMGGWSSKPRQGMGTNLSVPNPLGFFPDHQLDPAFGANSNNPDWDFNPNKDHWPEANQVGAGAFGPGFTPPHGGLLGWSPQAQGILTTVPAAPPPASTNRQSGRQPTPIELTRAPDYHGQDSVRVGRISFGSINAILGSVALILNCHHHASRVAREFGSELEAAAAKEAAAAKEAAAAKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 56 - protein, 6 aa, synthetic construct
  REGION 1..6: note = Rigid linker (n=1)
EAAAAK

(19) United States
(12) Patent Application Publication
(10) Pub. No.: US 2024/0409608 A1
(43) Pub. Date: Dec. 12, 2024

(54) COMBINATION THERAPIES AGAINST CANCER AND INFECTIOUS DISEASES

(71) Applicant: NAVICURE BIOPHARMACEUTICALS LIMITED, Apia City (WS)

(72) Inventor: Chia-Mao WU, Hsinchu City (TW)

(21) Appl. No.: 18/697,859

(22) PCT Filed: Sep. 19, 2022
(86) PCT No.: PCT/US2022/076636

§ 371 Date: Apr. 2, 2024

Related U.S. Application Data
(60) Provisional application No. 63/271,982, filed on Oct. 26, 2021.

(51) Int. Cl. C07K 14/705; A61K 39/00; A61K 39/12; A61P 31/20; A61P 35/00; C07K 14/005; C07K 16/28

(52) U.S. Cl. CPC C07K 14/70575; A61K 39/0011; A61K 39/12; A61P 31/20; A61P 35/00; C07K 14/005; C07K 16/2818; A61K 2039/505; A61K 2039/572; C07K 2317/76; C07K 2319/50; C07K 2319/55; C12N 2710/20022; C12N 2710/20034

(57) ABSTRACT

Combination immunotherapies against cancer and infectious diseases. A combination is disclosed, which comprises an immune checkpoint antibody and a fusion protein. The fusion protein comprises: a CD40-binding domain, an antigen, a translocation domain located between the CD40-binding domain and the antigen, and a furin and/or cathepsin L cleavage site located between the CD40-binding domain and the translocation domain. The antigen is an antigen of a pathogen or a tumor antigen. The furin and/or cathepsin L cleavage site permits removal of the CD40-binding domain away from the fusion protein. The combination of the invention is effective in eliciting an antigen-specific cell-mediated immune response, which is useful for treating a tumor and/or a disease caused by a pathogen in a subject in need thereof.

### REFERENCE TO AN ELECTRONIC SEQUENCE LISTING

The contents of the electronic sequence listing (10040-002PCT_sequence listing_ST26.xml; size 79 KB; creation date Aug. 23, 2022) is herein incorporated by reference in its entirety.

### FIELD OF THE INVENTION

The present invention relates generally to combination therapies, and more specifically to combinations of immunogenic fusion proteins with immune checkpoint antibodies for eliciting T cell-mediated immune responses against tumors and infectious diseases.

### BACKGROUND OF THE INVENTION

The adaptive immune system includes both humoral and cell-mediated immunity, both of which contribute to destroy invading pathogens. B- and T-lymphocytes are responsible for antibody and cell-mediated immune responses, respectively. Adaptive immunity against cancer or pathogens leads to enhanced immune responses to future encounters. Several adaptive immunity therapy strategies have been evaluated in the clinical settings. There is, however, still a need for developing new immunotherapies, e.g., combination immunotherapies, to combat cancer and infectious diseases caused by pathogens.

### SUMMARY OF THE INVENTION

In one aspect, the invention relates to a combination or pharmaceutical composition comprising: (a) an immune checkpoint antibody capable of activating T cell; and (b) a fusion protein, wherein the fusion protein comprises: (i) a CD40-binding domain; (ii) an antigen; (iii) a translocation domain, located between the CD40-binding domain and the antigen; and (iv) a furin and/or cathepsin L cleavage site, located between the CD40-binding domain and the translocation domain.

In another aspect, the invention relates to a combination or a pharmaceutical composition for use in eliciting an antigen-specific cell-mediated immune response, treating a tumor or a disease caused by a pathogen in a subject in need thereof.

### BRIEF DESCRIPTION OF DRAWINGS

FIGS. 1-4 are vector maps.

FIGS. 5A-E are schematic drawings illustrating various embodiments of the invention.

FIG. 6 is a graph showing relative cytokine inductions in each animal group.

FIG. 7 is a graph showing IFN-γ⁺ immunospots results of the splenocytes from each animal group.

FIG. 8 is a graph showing serum HPV₁₆ E7-specific antibody level in each animal group.

FIG. 9 is a graph showing serum HPV₁₈ E7-specific antibody level in each animal group.

FIG. 10 shows an immunization schedule (top panel) and animal groups treated with PD-1, PD-1 combined with fusion proteins, or placebo (bottom panel).

FIG. 11 is a graph showing tumor size in animal groups treated with PD-1, PD-1 combined with fusion proteins, or placebo.

FIG. 12 is a graph showing survival rate in animal groups treated with PD-1, PD-1 combined with fusion proteins, or placebo.

FIG. 13 is a graph showing tumor free rate in animal groups treated with PD-1, PD-1 combined with fusion proteins, or placebo.

FIG. 14 is a graph showing tumor size in animal groups treated with a fusion protein, CD137 mAb, CD137 mAb combined with the fusion protein, or placebo.

FIG. 15 is a graph showing survival rate in animal groups treated with a fusion protein, CD137 mAb, CD137 mAb combined with the fusion protein, or placebo.

### DETAILED DESCRIPTION OF THE INVENTION

### Definitions

Professional APCs and non-professional APCs use an MHC class I molecule to display endogenous peptides on the cell membrane. These peptides originate within the cell itself, in contrast to the exogenous antigen displayed by professional APCs using MHC class II molecules. Cytotoxic T cells are able to interact with antigens presented by the MHC class I molecule.

CD40 is a costimulatory protein expressed on antigen-presenting cells (e.g., dendritic cells, macrophages and B cells). The binding of CD40L to CD40 activates antigen-presenting cells and induces a variety of downstream effects. CD40 is a drug target for cancer immunotherapy.

The term “a CD40-binding domain” refers to a protein that can recognize and binds to CD40. A CD40-binding domain may be selected from one of the following: “CD40 ligand (CD40L) or a functional fragment thereof”, “an anti-CD40 antibody or a functional fragment thereof.”

The terms “CD40L”, “CD40 ligand” and “CD154” are interchangeable. CD40L binds to CD40 (protein) on antigen-presenting cells (APC), which leads to many effects depending on the target cell type. CD40L plays a central role in co-stimulation and regulation of the immune response via T cell priming and activation of CD40-expressing immune cells. U.S. Pat. No. 5,962,406 discloses the nucleotide and amino acid sequence of CD40L.

The terms “anti-CD40 antibody” and “CD40-specific antibody” are interchangeable.

When the term “consist substantially of” or “consisting substantially of” is used in describing an amino acid sequence of a polypeptide, it means that the polypeptide may or may not have a starting amino acid “M” (translated from a start codon AUG) at N-terminal as a part of the polypeptide, depending on protein translation requirements. For example, when the antigen HPV 18 E7 protein (SEQ ID NO: 39) fused to another polypeptide (e.g., another antigen), the starting amino acid “M” could be omitted or kept.

As used herein, “a translocation domain” is a polypeptide having biological activity in translocating an antigen within a fusion protein across an endosomal membrane into the cytosol of the CD40-expressing cell. The translocation domain guides or facilitates the antigen toward class I major histocompatibility complex (MHC-1) pathway (i.e., a cytotoxic T cell pathway) for antigen presentation.

The term “a Pseudomonas Exotoxin A (PE) translocation peptide (T^{PE})” refers to a PE domain II peptide or a functional fragment thereof that has the biological activity in translocation.

The term “a Shiga toxin (Stx) translocation peptide (T^{Stx})” refers to a Stx translocating domain or a functional fragment thereof that has the biological activity in translocation.

The terms “furin and/or cathepsin L” or “furin/cathepsin L” are interchangeable. A furin and/or cathepsin L cleavage site refers to a protease (furin and/or cathepsin L) sensitive site. It is a short peptide sequence that can be cleaved by furin or cathepsin L, or by both furin and cathepsin L. It may be a peptide linker comprising said cleavage site that is introduced into the fusion protein, or an intrinsic protease cleavage site present in the translocation domain of the fusion protein.

The terms “antigen” and “immunogen” are interchangeable. An antigen refers to an antigenic protein, which may be a tumor antigen (an antigen from a cancer or an antigen associated with a cancer), or an antigen of a pathogen (an antigen from a pathogen).

The terms “tumor” and “cancer” are interchangeable.

The terms “an antigen of a cancer cell” and “a tumor antigen” are interchangeable.

The term “a tumor antigen” refers to a tumor-specific antigen and/or a tumor-associated antigen. A tumor-associated antigen may be a protein or polypeptide expressed on the surface of a tumor cell.

Cluster of Differentiation 28 (CD28) is a T-cell-specific surface glycoprotein. A CD28 receptor is stimulated during the contact of T cells with antigen-presenting cells. Its function is involved in T-cell activation, the induction of cell proliferation and cytokine production and promotion of T-cell survival.

The term “an effective amount” refers to the amount of an active fusion protein that is required to confer a therapeutic effect on the treated subject. Effective doses will vary, as recognized by those skilled in the art, depending on rout of administration, excipient usage, and the possibility of co-usage with other therapeutic treatment.

The term “treating”, or “treatment” refers to administration of an effective amount of the fusion protein to a subject in need thereof, who has cancer or infection, or a symptom or predisposition toward such a disease, with the purpose of cure, alleviate, relieve, remedy, ameliorate, or prevent the disease, the symptoms of it, or the predisposition towards it. Such a subject can be identified by a health care professional based on results from any suitable diagnostic method.

The term “combination” or “combination therapy” or “combination immunotherapy” refers to a combination of one or more active drug substances that have a therapeutic effect. A combination may be provided in a single pharmaceutical composition so that a first active drug substance (e.g., an immune checkpoint antibody) and a second active drug substance (e.g., a fusion protein of the invention) can be administered together. In alternative embodiments, a combination may be provided using more than one pharmaceutical composition. In such embodiments, the first active drug substance may be provided in a first pharmaceutical composition and the second active drug substance may be provided in a second pharmaceutical composition so that the two drug substances can be administered separately such as, for example, at different times, by different routes of administration, and the like. Thus, it also may be possible to provide the two active drug substances in different dosing regimens.

By “0 to 12 repeats” or “2 to 6 repeats”, it means that all integer unit amounts within the range “0 to 12” or “2 to 6” are specifically disclosed as part of the invention. Thus, 0, 1, 2, 3, 4, . . . 10, 11 and 12″ or “2, 3, 4, 5 and 6” unit amounts are included as embodiments of this invention.

In one aspect, the invention relates to a combination comprising: (a) an immune checkpoint antibody capable of activating a T cell; and (b) a fusion protein, comprising: (i) a CD40-binding domain; (ii) an antigen; (iii) a translocation domain, located between the CD40-binding domain and the antigen; and (iv) a furin and/or cathepsin L cleavage site, located between the CD40-binding domain and the translocation domain.

An immune checkpoint antibody is able to restore or enhance T cell activation via (1) blocking an inhibitory immune checkpoint or (2) activating a stimulatory immune checkpoint.

In one embodiment, the immune checkpoint antibody is (1) an antagonist antibody capable of targeting an inhibitory immune checkpoint, (2) an agonist antibody capable of targeting a stimulatory immune checkpoint, or (3) a bispecific antibody capable of targeting two immune checkpoints. The inhibitory immune checkpoints include, but not limited to, PD-1, PD-L1, PD-L2, CTLA-4, LAG3, TIGIT, CD96, CD122R, TIM3, VISTA, CEACAMI, SIGLEC-7, SIGLEC-9, SIGLEC-15, KIRi, CD200R, BTLA, and ILT2. The stimulatory immune checkpoints include, but not limited to, CD137 (4-1BB), OX40, GITR, ICOS, CD27, CD28, CD40, KIRs, CD226 and CD244.

In some embodiments, the immune checkpoint antibody is an antagonist antibody capable of targeting PD-1, PD-L1, PD-L2, CTLA-4, LAG3, TIGIT, CD96, CD122R, TIM3, VISTA, CEACAMI, SIGLEC-7, SIGLEC-9, SIGLEC-15, KIRi, CD200R, BTLA, and ILT2. In some embodiments, the immune checkpoint antibody is an agonist antibody capable of targeting CD137 (4-1BB), OX40, GITR, ICOS, CD27, CD28, CD40, KIRs, CD226 and CD244.

In one embodiment, the immune checkpoint antibody is an anti-PD-1 antibody, e.g., Keytruda® (pembrolizumab), Opdivo® (nivolumab), Libtayo® (cemiplimab), or Jemperli® (dostarlimab). In one embodiment, the immune checkpoint antibody is an anti-PD-L1 antibody, e.g., Tecentriq® (atezolizumab), Imfinzi® (durvalumab), or Bavencio (avelumab). In one embodiment, the immune checkpoint antibody is an anti-CTLA-4 antibody, e.g., Yervoy® (ipilimumab). In one embodiment, the immune checkpoint antibody is an anti-LAG3 antibody, e.g., Relatlimab (BMS-986016). In one embodiment, the immune checkpoint antibody is an anti-TIGIT antibody, e.g., tiragolumab. In one embodiment, the immune checkpoint antibody is an anti-CD137 antibody, e.g., LVGN6051 or Urelumab (BMS-663513). In one embodiment, the immune checkpoint antibody is an anti-OX40 antibody, e.g., PF-04518600, BMS-986178, or MEDI6469. In one embodiment, the immune checkpoint antibody is a CD137/PD-L1 bispecific antibody, e.g., FS222. In one embodiment, the immune checkpoint antibody is a CD137/OX40 bispecific antibody, e.g., FS120.

In some embodiments, the immune checkpoint antibody comprises a fully antibody, a single chain variable fragment (scFv), a diabody (dscFv), a triabody, a tetrabody, a bispecific-scFv, a scFv-Fc, a scFc-CH3, a single chain antigen-binding fragment (scFab), an antigen-binding fragment (Fab), Fab2, a minibody, or an antibody analogue comprising one or more CDRs.

The fusion proteins of the invention can elicit an antigen-specific T cell immune response via MHC class I antigen presentation pathway. They share a common mechanism of action. Using 18sCD40L-T^{PE}-E7 as an example, the mechanism of action is as follows: (1) 18sCD40L-T^{PE}-E7 binds to a CD40-expressing cell (e.g., dendritic cell or macrophage) and is internalized via a CD40-mediated endocytosis; (2) 18sCD40L-T^{PE}-E7 is cleaved by furin protease and/or cathepsin L protease within the endosome so as to remove the 18sCD40L fragment away from the T^{PE}-E7 fragment; (3) the T^{PE}-E7 fragment is translocated across the endosomal membrane of the endosome into the cytosol; (4) the T^{PE}-E7 fragment is digested by cytosol proteasome to generate small E7 antigens with epitopes; (5) the E7 antigens are delivered via MHC class I pathway for antigen presentation; and (6) a CD8+ T cell specific immune response is induced or enhanced by T-cell recognizing these presented antigens.

The same mechanism of action is applicable to E7-T^{Stx}-18sCD40L, in which furin and/or cathepsin L protease cleavage would remove E7-T^{Stx} fragment away from 18sCD40L fragment. Thus, E7-T^{Stx} fragment would be translocated across the endosomal membrane of the endosome, enter the cytosol, digested by cytosol proteasome to generate small E7 antigens with epitopes; the small E7 antigens would be delivered via MHC class I pathway for antigen presentation; and a CD8+ T cell specific immune response would be induced or enhanced by T-cell recognizing these presented antigens.

No furin and/or cathepsin L cleavage site is present between the antigen and the translocation domain in the fusion protein of the invention. The presence of a furin and/or cathepsin L cleavage site and its location in the fusion protein of the invention permits removal of CD40-binding domain from the fusion protein after the furin and/or cathepsin L cleavage.

In some embodiments, the furin and/or cathepsin L cleavage site comprises or consists of 4-20 amino acids, preferred 4-10 amino acids, and more preferred 4-6 amino acids. In some embodiments, the furin and/or cathepsin L cleavage site comprises, consists of, or is, SEQ ID NO: 1 or 2.

The fusion protein of the invention may further comprise a peptide linker present between the CD40-binding domain and the translocation domain, wherein the furin and/or cathepsin L cleavage site is present in said peptide linker. The peptide linker may comprise (a) a rigid linker (EAAAAK)ₙ or (SEQ ID NO: 38)ₙ; and (b) a cleavable linker comprising a furin and/or cathepsin L cleavage site, wherein n is an integer from 0-12, preferably from 2-6, more preferably from 3-4, and said furin and/or cathepsin L cleavage site comprises SEQ ID NO: 1 or 2. In one embodiment, the peptide linker comprises (EAAAAK)₃ and RX¹RX²X³R (SEQ ID NO: 2; wherein X¹ is A, X² is Y, X³ is K). In another embodiment, the peptide linker comprises RX¹X²R (SEQ ID NO: 1; wherein X¹ is V, X² is A) and (EAAAAK)₃.

The translocation domain and the antigen are located within the fusion protein in such an orientation and/or relation that permits the translocating domain to translocate the antigen across the membrane of the endosome and enter the cytosol, and then facilitate the antigen toward MHC class I pathway for antigen presentation in the CD40-expressing cell.

The translocation domain may be derived from a Pseudomonas Exotoxin A (PE), or from a Shiga toxin (Stx). In one embodiment, the translocation domain comprises, or is, a Pseudomonas Exotoxin A (PE) translocation peptide (T^{PE}), with the proviso that the CD40-binding domain is located at the N-terminal of the fusion protein. In another embodiment, the translocation domain comprises, or is, a Shiga toxin (Stx) translocation peptide (T^{Stx}), with the proviso that the antigen is located at the N-terminal of the fusion protein.

In one embodiment, a fusion protein of the invention sequentially (from N-terminal to C-terminal) comprises: (a) a CD40-binding domain; (b) a furin and/or cathepsin L cleavage site; (c) a translocation domain comprising a PE translocation peptide (T^{PE}); and (d) an antigen.

In another embodiment, a fusion protein of the invention sequentially (from N-terminal to C-terminal) comprises: (a) a CD40-binding domain; (b) a peptide linker comprising a furin and/or cathepsin L cleavage site; (c) a translocation domain comprising a PE translocation peptide (T^{PE}); and (d) an antigen.

In another embodiment, a fusion protein of the invention sequentially (from N-terminal to C-terminal) comprises: (a) an antigen; (b) a translocation domain comprising a Stx translocation peptide (T^{Stx}); (c) a furin and/or cathepsin L cleavage site; and (d) a CD40-binding domain.

In another embodiment, a fusion protein of the invention sequentially (from N-terminal to C-terminal) comprises: (a) an antigen; (b) a translocation domain comprising a Stx translocation peptide (T^{Stx}); (c) a peptide linker comprising a furin and/or cathepsin L cleavage site; and (d) a CD40-binding domain.

The T^{PE} or T^{Stx} is a functional moiety having a biological activity in translocation. The furin and/or cathepsin L cleavage site may be one selected from SEQ ID NO: 1, SEQ ID NO: 2, or an intrinsic furin cleavage site within, or derived from, PE or Stx.

In one embodiment, a PE translocation peptide (T^{PE}) is domain II (amino acid residues 253-364; SEQ ID NO: 9) of Pseudomonas Exotoxin A protein (full-length PE, SEQ ID NO: 4) or a functional moiety thereof.

In some embodiments, the PE translocation peptide (T^{PE}) consists of 26-112 amino acid residues in length. The PE translocation peptide (T^{PE}) comprises a minimal functional fragment of GWEQLEQCGYPVQRLVALYLAARLSW (SEQ ID NO: 5).

In one embodiment, a PE translocation peptide (T^{PE}) comprises an amino acid sequence that is at least 95%, 97% or 99% identical to SEQ ID NO: 5, 6, 7, 8 or 9. In another embodiment, a T^{PE} comprises an amino acid sequence selected from the group consisting of SEQ ID NO: 5, 6, 7, 8 and 9. In another embodiment, a T^{PE} is PE₂₈₀₋₃₀₅ (SEQ ID NO: 5), PE₂₈₀₋₃₁₃ (SEQ ID NO: NO: 6), PE₂₆₈₋₃₁₃ (SEQ ID NO: NO: 7), PE₂₅₃₋₃₁₃ (SEQ ID NO: 8), or PE₂₅₃₋₃₆₄ (SEQ ID NO: 9; full-length PE domain II).

In one embodiment, a Stx translocation peptide (T^{Stx}) is a functional fragment of Shiga toxin (Stx) subunit A (SEQ ID NO: 10) or Shiga-like toxin I (Slt-I) subunit A (SEQ ID NO: 11). A Stx translocation peptide has translocation function but no cytotoxic effect of subunit A. Sequence identify between Shiga toxin (Stx) subunit A and Slt-I subunit A is 99% and the two proteins has only one amino acid difference.

In some embodiments, the Stx translocation peptide (T^{Stx}) consists of 8-84 amino acid residues in length. The Stx translocation peptide (T^{Stx}) comprises a minimal functional fragment of LNCHHHAS (SEQ ID NO: 12).

In one embodiment, a Stx translocation peptide (T^{Stk}) comprises an amino acid sequence that is at least 95%, 97% or 99% identical to SEQ ID NO: 12, 13, 14, 15 or 16. In another embodiment, a T^{Stx} comprises an amino acid sequence selected from the group consisting of SEQ ID NOs: 12, 13, 14, 15 and 16. In another embodiment, a T^{Stx} is Stx₂₄₀₋₂₄₇ (SEQ ID NO: 12), Stx₂₄₀₋₂₅₁ (SEQ ID NO: 13), Stx₂₁₁₋₂₄₇ (SEQ ID NO: 14), Stx₂₁₁₋₂₅₁ (SEQ ID NO: 15) or Stx₁₆₈₋₂₅₁ (SEQ ID NO: 16) of Stx subunit A.

A CD40-binding domain is a polypeptide having a biological activity in binding to CD40 protein on a CD40-expressing cell. A CD40-binding domain permits a fusion protein of the invention to bind to a CD40 receptor on a CD40-expressing cell (e.g., dendritic cell or macrophage). A CD40-binding domain may be one selected from the group consisting of (i) a CD40 ligand (CD40L) or a functional fragment thereof; and (ii) a CD40-specific antibody or a functional fragment thereof.

In one embodiment, a functional fragment of CD40L is a truncated CD40L having said biological activity, substantially lacking transmembrane and cytoplasmic regions of the full-length CD40L₁₋₂₆₁ protein (SEQ ID NO: 17).

In another embodiment, the CD40L or a functional fragment thereof consists of 154-261 amino acid residues in length. In some embodiments, the CD40L comprises a minimal functional fragment of SEQ ID NO: 19. In a particular embodiment, the CD40L or a functional fragment thereof consists of 154-261 amino acid residues in length and said CD40L comprises a minimal functional fragment of SEQ ID NO: 19.

In one embodiment, a CD40L comprises an amino acid sequence that is at least 95%, 97% or 99% identical to SEQ ID NO: 17, 18 or 19. In another embodiment, a CD40L is selected from the group consisting of CD40L₁₋₂₆₁ (SEQ ID NO: 17), CD40L₄₇₋₂₆₁ (SEQ ID NO: 18) and CD40L₁₀₈₋₂₆₁ (SEQ ID NO: 19).

In another embodiment, a CD40-binding domain is a CD40-specific antibody (or anti-CD40 antibody). A CD40-specific antibody is an antibody specifically recognizing and binding to CD 40 protein. A CD40-specific antibody can bind to CD40 protein on a CD40-expressing cell.

In one embodiment, the CD40-specific antibody comprises a heavy chain variable domain (V_{H}) and a light chain variable domain (V_{L}), wherein the V_{H} comprises the amino acid sequence of SEQ ID NO: 22; and the V_{L} comprises the amino acid sequence of SEQ ID NO: 23.

In another embodiment, the CD40-specific antibody is selected from the group consisting of a single chain variable fragment (scFv), a diabody (dscFv), a triabody, a tetrabody, a bispecific-scFv, a scFv-Fc, a scFc-CH3, a single chain antigen-binding fragment (scFab), an antigen-binding fragment (Fab), Fab2, a minibody and a fully antibody.

In another embodiment, a CD40-binding domain is a CD40-specific scFv (anti-CD40 scFv) comprising a heavy chain variable domain (V_{H}), a light chain variable domain (V_{L}) and a flexible linker (L) connecting the V_{H} and the V_{L}.

In one embodiment, a CD40-specific scFv comprises SEQ ID NO: 20 or 21.

In another embodiment, the CD40-binding domain according to the invention is (i) a CD40-specific antibody or a binding fragment thereof, or (ii) a CD40-specific single chain variable fragment (scFv) or a binding fragment thereof; said CD40-specific antibody or said CD40-specific scFv comprising a V_{H} and a V_{L} wherein: (a) the V_{H} comprises SEQ ID NO: 22; and (b) the V_{L} comprises SEQ ID NO: 23.

In another embodiment, the CD40-specific antibody or CD40-specific scFv comprises a V_{H} and a V_{L}, the V_{H} comprising V_{H} CDR1, V_{H} CDR2 and V_{H} CDR3; and the V_{L} comprising V_{L} CDR1, V_{L} CDR2 and V_{L} CDR3, wherein: (i) the V_{H} CDR1, V_{H} CDR2 and V_{H} CDR3 comprises SEQ ID NO: 24, 25 and 26, respectively; and (ii) the V_{L} CDR1, V_{L} CDR2 and V_{L} CDR3 comprises SEQ ID NO: 27, 28 and 29, respectively.

In another embodiment, the CD40-binding domain is a CD40-specific scFv comprising a V_{H} and a V_{L}, wherein: (a) the V_{H} comprises SEQ ID NO: 22; and (b) the V_{L} comprises SEQ ID NO: 23.

In another embodiment, the fusion protein of the invention further comprises an endoplasmic reticulum (ER) retention sequence located at the C-terminal of the antigen, with the proviso that the translocation domain comprises a PE translocation peptide (T^{PE}). As used herein, the ER retention sequence may comprise SEQ ID NO: 30, 31, 32, 33 or 34. In some embodiments, the ER retention is SEQ ID NO: 30.

In another embodiment, the fusion protein of the invention further comprises a CD28-activating peptide located between the CD40-binding domain and the furin and/or cathepsin L cleavage site.

In some embodiments, the CD28-activating peptide consists of 28-53 amino acid residues in length. In some embodiments, the CD28-activating peptide comprises a minimal functional fragment of SEQ ID NO: 35. In a particular embodiment, the CD28-activating peptide consists of 28-53 amino acid residues in length and said CD28-activating peptide comprises a minimal functional fragment of SEQ ID NO: 35.

In another embodiment, the CD28-activating peptide comprises an amino acid sequence that is at least 95%, 97% or 99% identical to SEQ ID NO: 35, 36 or 37. In another embodiment, the CD28-activating peptide comprises an amino acid sequence selected from the group consisting of SEQ ID NO: 35, 36 and 37. In another embodiment, the CD28-activating peptide is SEQ ID NO: 35, 36 or 37.

An antigen in the fusion protein of the invention is an antigen of a pathogen or a tumor antigen.

The pathogen may be selected from the group consisting of Human Papillomavirus (HPV), Human Immunodeficiency Virus-1 (HIV-1), Influenza Virus, Dengue Virus, Hepatitis A Virus (HAV), Hepatitis B Virus (HBV), Hepatitis C Virus (HCV), Hepatitis D Virus (HDV), Hepatitis E Virus (HEV), Severe Acute Respiratory Syndrome-Associated Coronavirus (SARS-COV), Severe Acute Respiratory Syndrome Coronavirus2 (SARS-COV2), Middle East Respiratory Syndrome Coronavirus (MERS-Cov), Epstein-Barr Virus (EBV), Zika Virus, Rabies Virus, Variola Virus, Chikungunya Virus, West Nile Virus, Poliovirus, Measles Virus, Rubella Virus, Hantavirus, Japanese Encephalitis Virus, Coxsackievirus, Echovirus, Enterovirus, Mumps Virus, Varicella-Zoster Virus (VZV), Cercopithecine Herpesvirus-1 (CHV-1), Yellow Fever Virus (YFV), Rift Valley Fever Virus, Lassa Virus, Marburg Virus, Ebolavirus, Norovirus, Rotavirus, Adenovirus, Sapovirus, Astrovirus, Porcine Reproductive and Respiratory Syndrome Virus (PRRSV), African Swine Fever Virus (ASFV), Classical Swine Fever Virus (CSFV), Porcine Circovirus 2 (PCV2), Foot-and-Mouth Disease Virus (FMDV), Porcine Epidemic Diarrhea Virus (PEDV), Swine Vesicular Disease Virus (SVDV), Pseudorabies Virus (PRV), Transmissible Gastroenteritis Virus (TGEV), Newcastle Disease Virus (NDV), Infectious Bronchitis Virus (IBV), Infectious Bursal Disease Virus (IBDV), Mycoplasma hyopneumoniae, Rickettsia prowazekii, Rickettsia typhi, Orientia tsutsugamushi, Borrelia burgdorferi, Yersinia pestis, Plasmodium vivax, Plasmodium malariae, Plasmodium falciparum, Plasmodium ovale, Bacillus anthracis, Clostridium Difficile, Clostridium Botulinum, Corynebacterium diphtheriae, Salmonella enterica serovar Typhi, Salmonella enterica scrovar Paratyphi A, Shiga toxin-producing E. coli (STEC), Shigella dysenteriae, Shigella flexneri, Shigella boydii, Shigella sonnei, Entamoeba histolytica, Vibrio cholerae, Mycobacterium tuberculosis, Neisseria meningitidis, Bordetella pertusis, Haemophilus influenzae type B (HiB), Clostridium tetani, Listeria monocytogenes and Streptococcus pneumoniae.

In another embodiment, the pathogen is selected from the group consisting of HPV, HIV-1, Influenza Virus, Dengue Virus, HAV, HBV, HCV, SARS-COV, SARS-COV-2. More particularly, the pathogen is selected from the group consisting of HPV, HBV, HCV and SARS-COV2.

In another embodiment, the antigen is a pathogenic antigen selected or derived from the group consisting of HPV 16 E7 protein, HPV₁₈ E7 protein, HBV X protein (HBx), HBV preS1 protein, HCV core protein (HCVcore), SARS-COV2 spike protein (CoV2S), SARS-COV2 envelope protein, SARS-CoV2 membrane protein and SARS-COV2 nucleocapsid protein.

In another embodiment, said antigen comprises at least one epitope for inducing a desired immune response, preferably containing 1 to 50 epitopes, more preferably containing 1 to 20 epitopes.

In another embodiment, the antigen is a pathogenic antigen comprising or consisting substantially of an amino acid sequence that is at least 70%, 80%, 90%, 95% or 99% identical to SEQ ID NO: 38, 39, 40, 41, 42 or 43.

In another embodiment, the antigen is a pathogenic antigen comprising or consisting substantially of an amino acid sequence that is at least 80% identical to SEQ ID NO: 38, 39, 40, 41, 42 or 43.

In another embodiment, the antigen comprises an amino acid sequence selected from the group consisting of SEQ ID Nos: 38, 39, 40, 41, 42 and 43.

In another embodiment, the antigen is a tumor antigen. A tumor antigen is a tumor-associated antigen (TAA) or a tumor-specific antigen (TSA).

In one embodiment, the tumor or cancer is selected from the group consisting of breast cancer, colon cancer, rectal cancer, bladder cancer, endometrial cancer, kidney cancer, gastric cancer, glioblastoma, hepatocellular carcinoma, bile duct cancer (cholangiocarcinoma), small cell lung cancer, non-small cell lung cancer (NSCLC), melanoma, ovarian cancer, cervical cancer, pancreatic cancer, prostate cancer, acute myelogenous leukemia (AML), chronic myelogenous leukemia (CML), non-Hodgkin's lymphoma, and thyroid cancer.

In another embodiment, a tumor-associated antigen is selected or derived from the group consisting of SSX2, MAGE-A3, NY-ESO-1, iLRP, WT12-281, RNF43, CEA-NE3, AFP, ALK, Anterior gradient 2 (AGR2), BAGE proteins, β-catenin, brc-abl, BRCA1, BORIS, CA9, carbonic anhydrase IX, caspase-8, CD40, CDK4, CEA, CTLA4, cyclin-B1, CYPIB1, EGFR, EGFRvIll, ErbB2/Her2, ErbB3, ErbB4, ETV6-AML, EphA2, Fra-1, FOLR1, GAGE proteins (e.g., GAGE-1, -2), GD2, GD3, GloboH, glypican-3, GM3, gp100, HLA/B-raf, HLA/k-ras, HLA/MAGE-A3, hTERT, LMP2, MAGE proteins (e.g., MAGE-1, -2, -3, -4, -6, and -12), MART-1, mesothelin, ML-IAP, Muc1, Muc16 (CA-125), MUM1, NA17, NY-BR1, NY-BR62, NY-BR85, NY-ES01, OX40, p15, p53, PAP, PAX3, PAX5, PCTA-1, PLAC1, PRLR, PRAME, PSMA (FOLH1), RAGE proteins, Ras, RGS5, Rho, SART-1, SART-3, Steap-1, Steap-2, survivin, TAG-72, TGF-β, TMPRSS2, Tn, TRP-1, TRP-2, tyrosinase, and uroplakin-3.

In another embodiment, the antigen is a tumor-associated antigen selected or derived from the group consisting of SSX2, MAGE-A3, NY-ESO-1, iLRP, WT12-281, RNF43 and CEA-NE3.

In another embodiment, the antigen is a tumor-associated antigen comprising an amino acid sequence that is at least 70%, 80%, 90%, 95% or 99% identical to SEQ ID NO: 44, 45, 46, 47, 48, 49 or 50.

In another embodiment, the antigen is a tumor-associated antigen comprising an amino acid sequence selected from the group consisting of SEQ ID NOs: 44, 45, 46, 47, 48, 49 and 50.

An antigen may be a single antigen or an antigenic fragment thereof, or a fusion antigen comprising at least two antigenic polypeptides fused together. For example, an antigen may be a single antigen of HPV₁₆ E7 protein or a fusion antigen comprising HPV₁₆ E7 and HPV₁₈ E7 proteins. A fusion antigen may or may not have a linker connecting different antigenic polypeptides.

In another embodiment, the antigen is a fusion antigen having at least one linker connecting different antigens.

In another embodiment, the antigen is a fusion antigen having a rigid linker, (EAAAAK)ₙ, connecting different antigens, wherein n is an integer from 0-12, preferably from 2-6, more preferably from 3-4. In other words, the rigid linker comprises 0 to 12 repeats, 2 to 6 repeats or 3-4 repeats of the sequence EAAAAK (SEQ ID NO: 56).

In another embodiment, the fusion protein of the invention further comprises a rigid linker between the CD40-binding domain and the furin and/or cathepsin L cleavage site. The rigid linker may be a peptide liner comprising 0 to 12 repeats of the amino acid sequence EAAAAK (SEQ ID NO: 56).

The rigid linker may be (EAAAAK)ₙ, or (SEQ ID NO: 56)ₙ, wherein n is an integer from 0-12, preferably from 2-6, more preferably from 3-4.

In another embodiment, the rigid linker comprises 2 to 6 repeats or 3-4 repeats of SEQ ID NO: 56.

In another embodiment, the fusion protein of the invention comprises, or consists substantially of, an amino acid sequence that is at least 90%, 95% or 99% identical to SEQ ID NO: 51, 52, 53, 54 or 55.

Further in another embodiment, the fusion protein of the invention comprises, or consists substantially of, an amino acid sequence selected from the group consisting of SEQ ID NOs: 51, 52, 53, 54 and 55.

The invention also relates to a pharmaceutical composition comprising: (a) a combination according to the invention; and (b) a pharmaceutical acceptable carrier or adjuvant.

The term “pharmaceutically acceptable carrier” includes any and all solvents, dispersion media, coatings, surfactants, antioxidants, preservatives, isotonic agents, absorption delaying agents, salts, drugs, drug stabilizers, binders, excipients, disintegration agents, lubricants, sweetening agents, flavoring agents, dyes, and combinations thereof, as would be known to one of ordinary skill in the art (see, for example, Remington's Pharmaceutical Sciences, 18th Ed. Mack Printing Company, 1990, pp. 1289-1329, incorporated herein by reference). Except in so far as any conventional carrier is incompatible with the active ingredient, its use in the therapeutic or pharmaceutical compositions is contemplated.

Suitable adjuvants include, but not limited to, a saponin-based adjuvant and a Toll-like receptor (TLR) agonist adjuvant. The saponin-based adjuvant may be GPI-0100, Quil A or QS-21. The TLR agonist adjuvant may be selected from a TLR3, TLR4 or TLR9 agonist, e.g., Poly I: C (TLR3 agonist), monophosphoryl lipid A (MPL; TLR4 agonist) or CpG oligonucleotide (TLR9 agonist). The CpG oligonucleotide adjuvant includes, but not limited to, class A CpG (i.e., CpG1585, CpG2216 or CpG2336), class B CpG (i.e., CpG1668, CpG1826, CpG2006, CpG2007, CpG BW006 or CpG D-SL01) and class C CpG (i.e., CpG2395, CpG M362 or CpG D-SL03). In addition, another adjuvant CpG1018 (Dynavax) is also considerable. In some embodiments, the adjuvant is a CpG oligonucleotide.

The pharmaceutical composition may be an enteral or a parenteral dosage form, suitable for transdermal, transmucosal, nasopharyngeal, pulmonary or direct injection, or for systemic (e.g., parenteral) or local (e.g., intratumor or intralesional injection) administration. Parenteral injection may be via intravenous (i.v.), intraperitoneal (i.p.), intramuscular (i.m.), subcutaneous (s.c.) or intradermal (i.d.) routes.

The pharmaceutical composition may also be administered orally, e.g., in the form of tablets, coated tablets, dragées, hard and soft gelatine capsules.

The dosage of the fusion protein may vary, depending on the disease to be controlled, the age and the individual condition of the patient and the mode of administration. The dosage may be fitted to individual requirements in each particular case so as to obtain a therapeutically effective amount of the fusion protein of the invention to achieve a desired therapeutic response.

For adult patients receiving the fusion protein provided herein, a single dosage of about 0.1 to 50 mg, especially about 0.1 to 5 mg, comes into consideration. Depending on severity of the disease and the precise pharmacokinetic profile, the fusion protein may be administered with one dosage unit per week, bi-week or month, and totally give 1 to 6 dosage units per cycle to satisfy such treatment.

The invention relates to a combination or a pharmaceutical composition and use thereof for eliciting an antigen-specific cell-mediated immune response, treating a tumor, or a disease caused by a pathogen in a subject in need thereof.

The invention further relates to use of a combination or a pharmaceutical composition in the manufacture of a medicament for eliciting an antigen-specific cell-mediated immune response, treating a tumor or a disease caused by a pathogen in a subject in need thereof. The invention also relates to use of an immune checkpoint antibody in combination with use of a fusion protein defined above in the manufacture of medicament(s) for eliciting an antigen-specific cell-mediated immune response.

Alternatively, the invention relates to a method for eliciting an antigen-specific cell-mediated immune response in a subject in need thereof, comprising administering an effective amount of the combination or the pharmaceutical composition of the invention to the subject in need thereof.

A combination or a composition of the invention is useful for treating a tumor or an abnormal cell proliferation, or a disease caused by a pathogen in a subject in need thereof. The abnormal cell proliferation may be a pre-cancerous lesion or a tumor.

To administer a combination or a composition comprising an immune checkpoint antibody and a fusion protein, the antibody and the fusion protein may be administered to a patient simultaneously in the form of a single entity or dosage or administered to a patient as separate entities either simultaneously or sequentially. Such administration provides therapeutically effective amounts of active ingredients to the patient. For example, a fusion protein in a dose of between about 0.01 mg/kg, 0.02 mg/kg, 0.025 mg/kg, 0.05 mg/kg, 0.075 mg/kg, 0.1 mg/kg, 0.125 mg/kg. 0.15 mg/kg, 0.175 mg/kg, 0.2 mg/kg, 0.225 mg/kg, 0.25 mg/kg, 0.5 mg/kg, 0.75 mg/kg, 1 mg/kg, 1.25 mg/kg, 1.5 mg/kg, 1.75 mg/kg, 2 mg/kg, 2.25 mg/kg, 2.5 mg/kg, 2.75 mg/kg, 3 mg/kg, 3.25 mg/kg, 3.5 mg/kg, 3.75 mg/kg, 4 mg/kg, 4.25 mg/kg, 4.5 mg/kg, 4.75 mg/kg, to about 5 mg/kg all inclusive, twice daily, once daily, once every two, three, four, five or six days, or once, twice, or three times per week.

In another aspect, the invention provides kits containing the therapeutic combinations and directions for use thereof.

Abbreviations: MCS, multiple cloning sites; anti-PD-1, anti-programmed cell death-1; anti-PD-L₁, anti-programmed cell death ligand-1; Rap1, Ras-proximate-1 or Ras-related protein J; CD40, Cluster of differentiation 40; CDR, Complementarity-determining region.

### EXAMPLES

### Methods and Materials

Table 1 shows SEQ ID numbers of corresponding peptides, polypeptides and fusion proteins.

TABLE 1

| SEQ ID | | Length |
|---|---|---|
| No. | Component name or sequence (N→C) | (aa) |
| 1 | Cleavable linker 1 | 4 |
| | RX¹X²R, wherein X¹ and X² are any amino acid residue. | |
| 2 | Cleavable linker 2 | 6 |
| | RX¹RX²X³R, wherein X¹ and X² are any amino acid | |
| | residue, and X³ is K, F or R. | |
| 3 | Rigid linker 1 (EAAAAK)₃ | 18 |
| 4 | Full length PE | 613 |
| 5 | PE translocation peptide (PE₂₈₀₋₃₀₅, minimal) | 26 |
| 6 | PE translocation peptide (PE₂₈₀₋₃₁₃) | 34 |
| 7 | PE translocation peptide (PE₂₆₈₋₃₁₃) | 46 |
| 8 | PE translocation peptide (PE₂₅₃₋₃₁₃) | 61 |
| 9 | PE translocation peptide (PE₂₅₃₋₃₆₄) | 112 |
| 10 | Full length Shiga toxin (Stx) subunit A | 293 |
| 11 | Full length Shiga-like toxin I (Slt-I) subunit A | 293 |
| 12 | Stx translocation peptide (Stx₂₄₀₋₂₄₇, minimal) | 8 |
| 13 | Stx translocation peptide (Stx₂₄₀₋₂₅₁) | 12 |
| 14 | Stx translocation peptide (Stx₂₁₁₋₂₄₇) | 37 |
| 15 | Stx translocation peptide (Stx₂₁₁₋₂₅₁) | 41 |
| 16 | Stx translocation peptide (Stx₁₆₈₋₂₅₁) | 84 |
| 17 | Full length CD40 ligand (CD40L₁₋₂₆₁) | 261 |
| 18 | Truncated CD40 ligand (CD40L₄₇₋₂₆₁) | 215 |
| 19 | Truncated CD40 ligand (CD40L₁₀₈₋₂₆₁, also referred to | 154 |
| | as 18sCD40L) | |
| 20 | Anti-CD40 scFv (V_{H}-L-V_{L}) | 246 |
| 21 | Anti-CD40 scFv (V_{L}-L-V_{H}) | 246 |
| 22 | V_{H} of anti-CD40 scFv | 119 |
| 23 | V_{L} of anti-CD40 scFv | 112 |
| 24 | V_{H} CDR1 GFTFSTYGMH | 10 |
| 25 | V_{H} CDR2 GKGLEWLSYISGGSSYIFYADSVRGR | 26 |
| 26 | V_{H} CDR3 CARILRGGSGMDL | 13 |
| 27 | V_{L} CDR1 CTGSSSNIGAGYNVY | 15 |
| 28 | V_{L} CDR2 GNINRPS | 7 |
| 29 | V_{L} CDR3 CAAWDKSISGLV | 12 |
| 30 | ER retention sequence KDEL | 4 |
| 31 | ER retention sequence KKDLRDELKDEL | 12 |
| 32 | ER retention sequence KKDELRDELKDEL | 13 |
| 33 | ER retention sequence KKDELRVELKDEL | 13 |
| 34 | ER retention sequence KDELKDELKDEL | 12 |
| 35 | CD28 consensus sequence | 28 |
| | T¹D²I³Y⁴F⁵C⁶K⁷X⁸E⁹X¹⁰X¹¹Y¹²P¹³P¹⁴P¹⁵Y¹⁶X¹⁷D¹⁸N¹⁹ | |
| | E²⁰K²¹S²²N²³G²⁴T²⁵I²⁶I²⁷H²⁸, wherein X⁸ is I or L, | |
| | X¹⁰ is V, For A, X¹¹ is M or L, X¹⁷ is L or I. | |
| 36 | CD28-activating peptide (minimal) | 28 |
| 37 | CD28-activating peptide | 53 |
| 38 | Antigen HPV₁₆ E7 protein | 98 |
| 39 | Antigen HPV₁₈ E7 protein | 104 |
| 40 | Antigen HBV X protein (HBx; full length) | 154 |
| 41 | Antigen HBV preS1 protein | 108 |
| 42 | Antigen HCV core protein (full length) | 190 |
| 43 | Antigen SARS-COV2 spike protein | 1273 |
| 44 | Antigen SSX2 | 187 |
| 45 | Antigen MAGE-A3 | 314 |
| 46 | Antigen NY-ESO-1 | 180 |
| 47 | Antigen iLRP | 296 |
| 48 | Antigen WT12-281 | 279 |
| 49 | Antigen RNF43 | 406 |
| 50 | Antigen CEA-NE3 | 284 |
| 51 | Fusion protein CD40L₄₇₋₂₆₁-T^{PE}-E7 | 528 |
| 52 | Fusion protein 18sCD40L-T^{PE}-E7 | 467 |
| 53 | Fusion protein E7-T^{Stx}-CD40L₄₇₋₂₆₁ | 535 |
| 54 | Fusion protein E7-T^{Stx}-18sCD40L | 474 |
| 55 | Fusion protein HBx-preS1-T^{Stx}-18sCD40L | 541 |
| 56 | Rigid linker EAAAAK (n = 1) | 6 |

Flow cytometry. Splenocytes were stimulated with an antigenic stimulator for 2 hours at 37° C., followed by treating with 50 μg/mL of Brefeldin A and Monensin at 37° C. for 2 hours. The cells were harvested, washed with PBS containing 0.5% BSA, and stained with APC/Cy7-conjugated anti-CD3 antibody, PerCP/Cy5.5-conjugated anti-CD4 antibody, FITC-conjugated anti-CD8 antibody, PE-conjugated anti-CD44 antibody and APC-conjugated anti-CD62L antibody simultaneously. After wash, the cells were permeabilized, fixed and intracellularly stained with PE-conjugated anti-IFN-γ antibody, PE/Cy7-conjugated anti-IL-2 antibody and eFluor450-conjugated anti-TNF-α antibody simultaneously. The intracellular cytokine characterization (IFN-γ, IL-2 or TNF-α) of splenocytes with CD8+ or CD4+ memory T cell phenotypes (CD3⁺/CD44^{hi}CD62^{Lo}) were further analyzed by Gallios flow cytometer and Kaluza software.

Enzyme-linked immunospot (ELISpot) assay. Splenocytes were seeded in triplicate in a pretreated murine IFN-γ capturing 96-well plate (CTL IMMUNOSPOT) at a cell density of 2×10⁵ cells/well in the presence or absence of an antigenic stimulator. The cells were discarded after 24 hours of incubation at 37° C. After wash, the captured IFN-γ was detected by biotin-conjugated anti-murine IFN-γ antibody at room temperature for 2 hours and the IFN-γ-immunospots were developed according to the manufacturer's instructions. The scanning and counting of IFN-γ-immunospots was performed by IMMUNOSPOT® S5 Micro analyzer (CTL).

Indirect enzyme-linked immunosorbent assay (ELISA). Collected whole blood samples were left undisturbed at 4° C. for 30-60 minutes followed by centrifugation at 5,000 g for 10 minutes to pellet the clot. The serum samples were stored at −20° C. The purified coating protein for antigen-specific antibody binding was diluted in guanidine coating buffer (2 M guanidine hydrochloride, 500 mM Na₂HPO₄, 25 mM citrate, pH 4.0-4.4) and distributed into 96-well plate at 1 μg/well. After overnight incubation at 4° C., the 96-well plate was blocked with 1% BSA in PBS at 37° C. for 1 hour. The serum samples were thawed, and subsequently 10-fold serial diluted in PBS with 1% BSA. The coated protein was incubated with 100 μl of 1000-fold diluted serum sample at 37° C. for 2 hours. After 4 times washing with phosphate buffered saline TWEEN®-20 (PBST), the antigen-specific antibodies were detected by horseradish peroxidase (HRP)-conjugated goat anti-mouse IgG (at a dilution of 1:10,000, Cat #31430, Thermo Fisher Science) at 37° C. for 30 minutes. Following 4 times of washing with PBST, the HRP-mediated color development was catalyzed in the presence of 100 μL of TMB substrate and quenched by 100 μL of 1 N HCl. The relative titers of antigen-specific antibody in the serum samples were determined by the absorbance at 450 nm.

Statistical analysis. Using a t-test, results considered significant when p<0.05.

### Example 1

### Construction of Expression Vectors

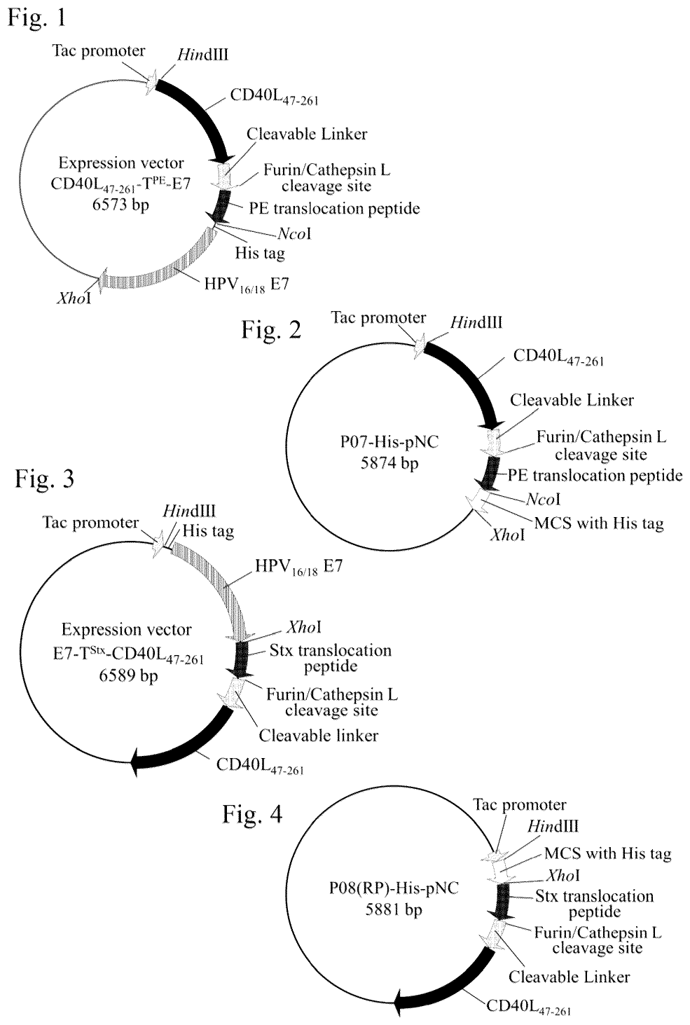

CD40L₄₇₋₂₆₁-T^{PE}-E7 and 18sCD40L-T^{PE}-E7. The vector CD40L₄₇₋₂₆₁-T^{PE}-E7 (FIG. 1) was constructed to generate CD40L₄₇₋₂₆₁-T^{PE}-E7 (SEQ ID NO: 51; FIG. 5A) fusion protein, which comprises: (a) a truncated CD40 ligand (CD40L₄₇₋₂₆₁; SEQ ID NO: 18); (b) a cleavable peptide linker, comprising (EAAAAK)₃ (SEQ ID NO: 3) and RX¹RX²X³R (SEQ ID NO: 2; wherein X¹ is A, X² is Y, X³ is K); (c) a PE translocation peptide (PE₂₈₀₋₃₀₅; SEQ ID NO: 5); and (d) a fusion antigen (HPV_{16/18} E7), comprising a HPV₁₆ E7 protein (SEQ ID NO: 38) and a HPV 18 E7 protein (SEQ ID NO: 39). Briefly, a DNA fragment encoding ^{HindIII}CD40L-Linker-PE^{NcoI,XhoI,SalI}, which comprises the CD40L₄₇₋₂₆₁, a cleavable linker and a PE translocation peptide (PE₂₈₀₋₃₀₅), was PCR synthesized, digested by HindIII/SalI and ligated into the plasmid pTAC-MAT-Tag-2 having HindIII/XhoI cutting sites to obtain the plasmid P07-His-pNC (FIG. 2). Another DNA fragment encoding an HPV_{16/18} E7 fusion antigen carrying a His tag was inserted into the plasmid PO7-His-pNC (FIG. 2) via NcoI/XhoI sites to generate the expression vector CD40L₄₇₋₂₆₁-T^{PE}-E7 (FIG. 1). The cleavable linker allows furin and/or cathepsin L protease to cut the fusion protein of the invention for releasing the T^{PE}-E7 fragment from the fusion protein.

Using a similar method described above, any other antigen(s) of interest may replace E7 and be inserted into the plasmid of FIG. 2 to generate an expression vector like FIG. 1 for expressing a fusion protein comprising the antigen of interest.

Similarly, an expression vector for generating 18sCD40L-T^{PE}-E7 fusion protein (SEQ ID NO: 52; FIG. 5B) was constructed, by replacing the truncated CD40 ligand CD40L₄₇₋₂₆₁ (SEQ ID NO: 18) with CD40L₁₀₈₋₂₆₁ (SEQ ID NO: 19; 18sCD40L), which is another truncated CD40 ligand.

E7-T^{Stx}-CD40L₄₇₋₂₆₁ and E7-T^{Stx}-18sCD40L. The vector E7-T^{Stx}-CD40L₄₇₋₂₆₁ (FIG. 3) was constructed to generate E7-T^{Stx}-CD40L₄₇₋₂₆₁ (SEQ ID NO: 53; FIG. 5C) fusion protein, which comprises: (a) a fusion antigen (HPV 16/18 E7), comprising a HPV 16 E7 protein (SEQ ID NO: 38) and a HPV 18 E7 protein (SEQ ID NO: 39); (b) a Stx translocation peptide (Stx₂₁₁₋₂₄₇; SEQ ID NO: 14); (c) a cleavable peptide linker, comprising RX¹X²R (SEQ ID NO: 1; wherein X¹ is V, X² is A) and (EAAAAK)₃ (SEQ ID NO: 3); and (d) a truncated CD40 ligand (CD40L₄₇₋₂₆₁; SEQ ID NO: 18).

Briefly, a DNA fragment encoding ^{HindIII,XhoI}Stx-Linker-CD40L^{SalI}, which comprises a Stx translocation peptide (Stx₂₁₁₋₂₄₇), a cleavable linker and CD40L₄₇₋₂₆₁, was PCR synthesized, digested by HindIII/Sa/I, ligated into the plasmid pTAC-MAT-Tag-2 having HindIII/XhoI cutting sites to obtain the plasmid P08(RP)-His-pNC (FIG. 4). Another DNA fragment encoding an HPV_{16/18} E7 fusion antigen carrying a His tag was inserted into the plasmid P08(RP)-His-pNC (FIG. 4) via HindIII/XhoI sites to generate the expression vector E7-T^{Stx}-CD40L₄₇₋₂₆₁ (FIG. 3).

The cleavable linker is vital because it allows the fusion protein of the invention to be cut by furin and/or cathepsin L protease inside a cell and release the E7-T^{Stx} fragment (FIG. 5C).

Any other antigen(s) of interest from various pathogen or cancer origins may replace E7 and be inserted into the plasmid of FIG. 4 to generate an expression vector similar to that in FIG. 3 to express a fusion protein comprising any antigen of interest.

Similarly, an expression vector for generating E7-T^{Stx}-18sCD40L fusion protein (SEQ ID NO: 54; FIG. 5D) was constructed, by replacing the truncated CD40 ligand CD40L₄₇₋₂₆₁ (SEQ ID NO: 18) with CD40L₁₀₈₋₂₆₁ (SEQ ID NO: 19; 18sCD40L), which is another truncated CD40 ligand.

For a comparison purpose, RAP1-CD28_{conv}PEₜ-E7-K3 fusion protein (named as “RAP1-E7”) was constructed. It comprised a RAP1 domain III, a CD28 sequence, a linker, a PE translocation domain II (PE₂₆₈₋₃₁₃), an antigen E7 protein and an endoplasmic reticulum retention sequence, in which the antigen E7 protein was a fusion antigen (HPV_{16/18} E7) comprising an HPV₁₆ E7 protein (SEQ ID NO: 38) and an HPV₁₈ E7 protein (SEQ ID NO: 39). This “RAP1-E7” fusion protein was almost identical to a prior construct disclosed in the U.S. Pat. No. 9,481,714 B2, Example 1, with the only difference in that the antigen E7 protein disclosed in the prior art was HPV₁₆ E7 protein, rather than an HPV_{16/18} E7 fusion antigen.

HBx-preS1-T^{Stx}-18sCD40L. The vector HBx-preS1-T^{Stx}-18sCD40L was constructed to generate HBx-preS1-T^{Stx}-18sCD40L fusion protein (SEQ ID NO: 55; FIG. 5E), which comprises: (a) a fusion antigen (HBx-preS1), comprising a HBx protein (SEQ ID NO: 40) and a HBV preS1 protein (SEQ ID No: 41); (b) a Stx translocation peptide (Stx₂₁₁₋₂₄₇; SEQ ID NO: 14); (c) a cleavable peptide linker, comprising RX¹X²R (SEQ ID NO: 1, wherein X¹ is V, X² is A) and (EAAAAK)₃ (SEQ ID NO: 3); and (d) a truncated CD40 ligand CD40L₁₀₈₋₂₆₁ (SEQ ID NO: 19; 18sCD40L).

The vector HBx-preS1-T^{Stx}-18sCD40L was constructed using a similar method as aforementioned, in which the truncated CD40 ligand was replaced by CD40L₁₀₈₋₂₆₁ and the fusion antigen was replaced by HBx-preS1.

### Example 2

### Protein Expression

E. coli BL21 cells harboring expression vectors were grown in ZY media (10 g/L tryptone and 5 g/L yeast extract) containing selection antibiotics at 37° C. When the culture reached an early log phase (OD₆₀₀=2 to 5), the expression of fusion protein was induced by isopropyl-1-thio-β-D-galactopyranoside (IPTG) (0.5 to 2 mM). Cells were harvested after 4 hours of IPTG induction and disrupted by sonication. The inclusion bodies were isolated and solubilized in solubilization buffer (6 M guanidine hydrochloride, 20 mM potassium phosphate, 500 mM NaCl, 20 mM imidazole, 1 mM DTT, pH 7.4) to recover overexpressed fusion proteins. After purification, the refolding of the fusion proteins was performed by dialysis against 20- to 50-fold volume of dialysis buffer (10 mM PBS) at 4° C. overnight. The refolded fusion proteins were subject to SDS-PAGE analyses under reduced (with dithiothreitol; +DTT) and non-reduced (without dithiothreitol; −DTT) conditions to evaluate whether they were properly refolded.

### Example 3

### Immunogenicity Analyses of Fusion Proteins

The fusion proteins CD40L₄₇₋₂₆₁-T^{PE}-E7, 18sCD40L-T^{PE}-E7, E7-T^{Stx}-18sCD40L and RAP1-E7 purified were further subjected to immunogenicity analyses to evaluate their biological activities.

Female C57BL/6NCrlBltw mice (5 to 6-week-old) were randomly divided into 5 groups (n=5): (A) placebo (i.e., PBS); (B) CD40L₄₇₋₂₆₁-T^{PE}-E7 (100 μg) fusion protein; (C) 18sCD40L-T^{PE}-E7 (100 μg) fusion protein; (D) E7-T^{Stx}-18sCD40L (100 μg) fusion protein; and (E) RAP1-E7 (100 μg) fusion protein. The fusion proteins were dialyzed into PBS. CpG1826 (50 μg) was used as an adjuvant in animal groups B to E. Each group received three immunizations subcutaneously (s.c.) at 7 day intervals from day 0. Blood samples were collected on day 0, 7 and 14. On day 21, the blood samples were harvested and the splenocytes were resuspended in RPMI 1640 medium containing FBS (10%) and PSA.

The splenocytes were used to analyze intracellular cytokine induction (IFN-γ, IL-2 and TNF-α) in the CD8 and CD4 memory T cells in the presence and absence of antigen stimulation. Briefly, splenocytes from each animal group were treated with or without antigen E7 protein (2 μg/mL of HPV₁₆ E7 peptide pool) and then analyzed by flow cytometry. The degree or the level of the intracellular cytokine induction in each mouse group was presented as a relative cytokine induction, which was obtained by normalizing the frequency of cytokine/CD8 and cytokine/CD4 splenocytes in the presence of the stimulating antigen E7 to that of the unstimulated (untreated) control.

The splenocytes were also used to analyze the frequency of IFN-γ-secreting splenocytes in the presence and absence of antigen stimulation (2 μg/mL of HPV₁₆ E7 peptide pool) by using Enzyme-linked immunospot (ELISpot) assay. The results were presented as IFN-γ⁺ immunospots per million splenocytes.

The blood samples were used to analyze the level of serum HPV₁₆ E7-specific and HPV₁₈ E7-specific antibody by using ELISA, in which the purified HPV₁₆ E7 and HPV₁₈ E7 recombinant proteins were used as coating proteins, respectively.

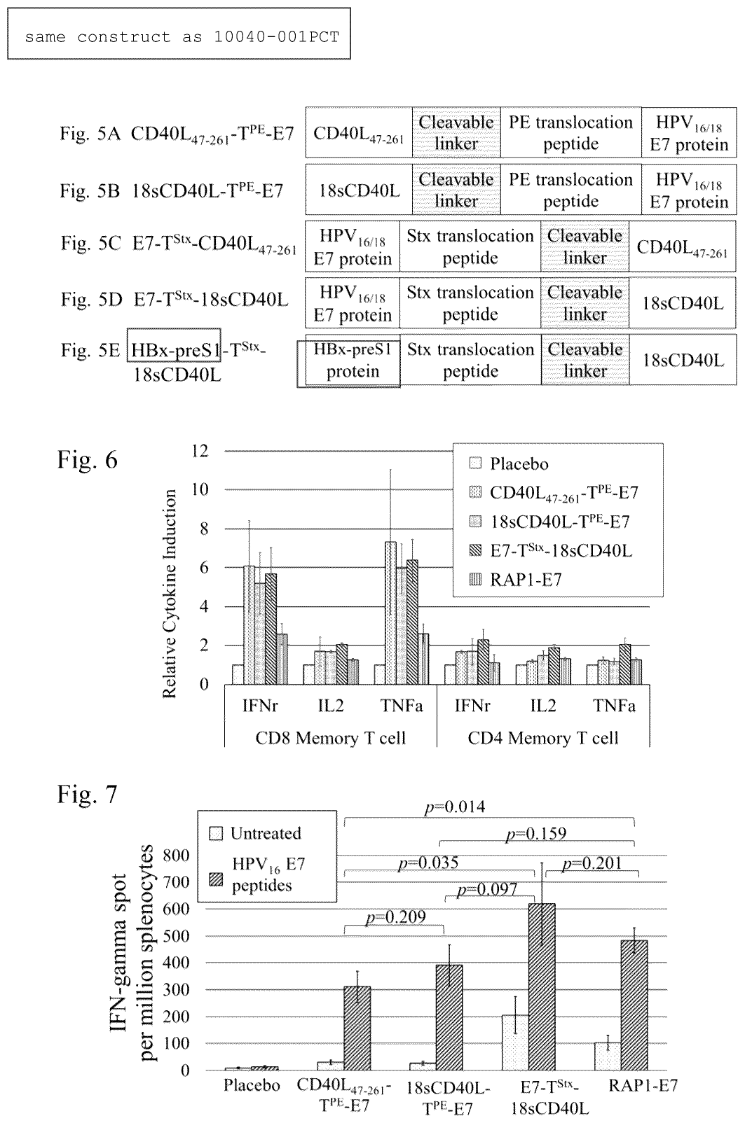

FIG. 6 shows cytokine induction after antigen stimulation of splenocytes with HPV₁₆ E7 peptide pool. The relative cytokine induction of IFN-γ and TNF-α, but not IL2, in CD8⁺ memory T cells from animals immunized with CD40L₄₇₋₂₆₁-T^{PE}-E7, 18sCD40L-T^{PE}-E7, or E7-T^{Stx}-18sCD40L fusion proteins showed a significant increase as compared to that from animals treated with RAP1-E7 fusion protein or placebo. The relative cytokine induction of IFN-γ, IL-2 or TNF-α in CD4 memory T cells from animal groups treated with fusion proteins showed a slight, but not significant, increase as compared to the placebo group.

It was concluded that the fusion protein of the invention was superior to the prior art fusion protein in inducing IFN-γ and TNF-α secretions in CD8 memory T cells in response to the stimulation of the antigen HPV₁₆ E7.

FIG. 7 shows IFN-γ immunospots of splenocytes stimulated with HPV₁₆ E7 peptide pool in vitro. The frequency of IFN-γ-secreting splenocytes from animal groups immunized with CD40L₄₇₋₂₆₁-T^{PE}-E7, 18sCD40L-T^{PE}-E7, E7-T^{Stk-18}sCD40L, or RAP1-E7 significantly increased as compared to the placebo group. Particularly, E7-T^{Stx}-18sCD40L induced significantly higher frequency of IFN-γ-secreting cells than CD40L₄₇₋₂₆₁-T^{PE}-E7 (p=0.035).

The results indicated that the fusion proteins of the invention could significantly increase IFN-γ-secreting T cell population upon or after stimulation with the antigenic HPV₁₆ E7 peptide pool.

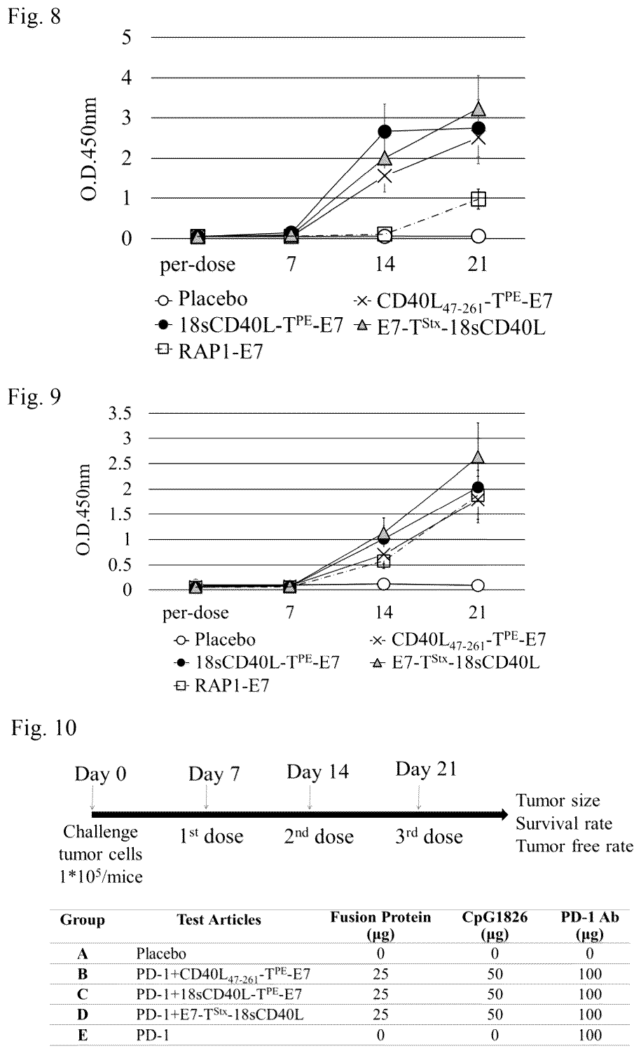

FIG. 8 shows the results of serum HPV₁₆ E7-specific antibody levels in animals immunized with various fusion proteins on day 0, 7 and 14. The serum HPV₁₆ E7-specific antibody level in animals vaccinated with CD40L₄₇₋₂₆₁-T^{PE}-E7, 18sCD40L-T^{PE}-E7, or E7-T^{Stx}-18sCD40L started to increase after the second vaccination on day 7, further rose after the third vaccination on day 14, and were higher than animals treated with placebo or RAP1-E7 on day 21.

RAP1-E7 (RAP1-CD28convPEt-E7-K3) fusion protein failed to elicit HPV₁₆ E7-specific antibody level after two vaccinations (on day 0 and 7). It started to induce serum HPV₁₆ E7-specific antibody after the third vaccination on day 14, and the serum antibody level was only modest on day 21 as compared to animals treated with the aforementioned fusion proteins of the invention. A similar pattern was also observed in inducing HBx-specific antibody when animals were vaccinated with RAP1-CD28convPEt-HBx-K3 (referred to as “RAP1-HBx”), using the same regimen and immunization schedule described above. The fusion protein RAP1-HBx was generated by using HBx antigen to replace the E7 antigen in the RAP1-E7. The fusion protein RAP1-HBx induced serum HBx-specific antibody level after the third vaccination on day 14, and the serum antibody level on day 21 was only modest (data not shown).

In contrast, the fusion protein of the invention elicited serum HPV₁₆ E7-specific antibody level after two shots of the vaccines on day 0 and 7 (FIG. 8).

FIG. 9 shows the serum HPV₁₈ E7-specific antibody level in animals immunized with various fusion proteins on day 0, 7 and 14. CD40L₄₇₋₂₆₁-T^{PE}-E7, 18sCD40L-T^{PE}-E7 and E7-T^{Stx}-18sCD40L fusion proteins significantly increased the serum HPV₁₈ E7-specific antibody level as compared to the placebo group. Immunogenicity analyses of HBx-preS1-T^{Stx}-18sCD40L fusion protein described above was performed. The results indicated that the HBx-carrying fusion protein could effectively elicit HBx-specific T cell-mediated and humoral immune responses after at least twice immunizations (data not shown).

Thus, the fusion proteins of the invention were effective in inducing antigen-specific antibodies and the antibody induction occurred after twice vaccinations.

In summary, the antigen-carrying fusion proteins of the invention could effectively induce antigen-specific T cell response, increase the expression of proinflammatory cytokines, e.g., IFN-γ and TNF-α, and generate antigen-specific antibody response.

### Example 4

### Efficacy Analyses

Fusion Proteins in Combination with an Immune Checkpoint Inhibitor in a Mouse Model of HPV Infection

CD40L₄₇₋₂₆₁-T^{PE}-E7, 18sCD40L-T^{PE}-E7, and E7-T^{Stx}-18sCD40L fusion proteins were each combined with an immune checkpoint inhibitor antibody (an anti-PD-1 antibody), and each combination was tested for efficacy in a mouse HPV 16 tumor model.

Female C57BL/6NCrlBltw mice (5 to 6-week-old) were randomly divided into 5 groups: (A) placebo (PBS, n=4); (B) anti-PD-1 antibody (100 μg; catalog no. BE0146, Bio X Cell, Inc.) in combination with CD40L+7-261-T^{PE}-E7 (25 μg) (n=5); (C) anti-PD-1 antibody (100 μg) in combination with 18sCD40L-T^{PE}-E7 (25 μg) (n=4); (D) anti-PD-1 antibody (100 μg) in combination with E7-T^{Stx}-18sCD40L (25 μg) (n=5); and (E) anti-PD-1 antibody alone (100 μg) (n=5). In Groups B to D, the fusion proteins were dissolved in PBS and CpG1826 (50 μg) was used as an adjuvant in immunizing animals. FIG. 10 shows an immunization schedule, combination therapies and the dosages.

An HPV₁₆ E6- and E7-expressing tumor cell line (TC-01) from lung epithelial cells of C57BL/6 mice was used to establish a mouse HPV 16 tumor model. Tumor cells were grown in RPMI 1640 medium containing FBS (10%) and penicillin/streptomycin/Amphotericin B (50 units/mL) at 37° C., 5% CO2. To challenge mice, tumor cells (1×10⁵ in 0.1 mL) were injected subcutaneously into the left flank of each mouse on day 0. Mice in each group received three doses of test articles (i.e., placebo, anti-PD-l antibody, or an combination therapy) on days 7, 14 and 21. In each dosing, the fusion protein was given subcutaneously, and the anti-PD-1 antibody was given intraperitoneally. All survived mice were sacrificed on day 39.

The tumor size was determined twice a week by multiplication of caliper measurements based on the modified ellipsoidal formula: Tumor volume=1/2 (length×width²). The survival rate and tumor free rate were calculated. Mice with tumor length over 2 cm were considered dead and mice without measurable or palpable tumor masses were considered tumor-free. The significance of each comparison was calculated by using t-test, and results considered significant when p<0.05.

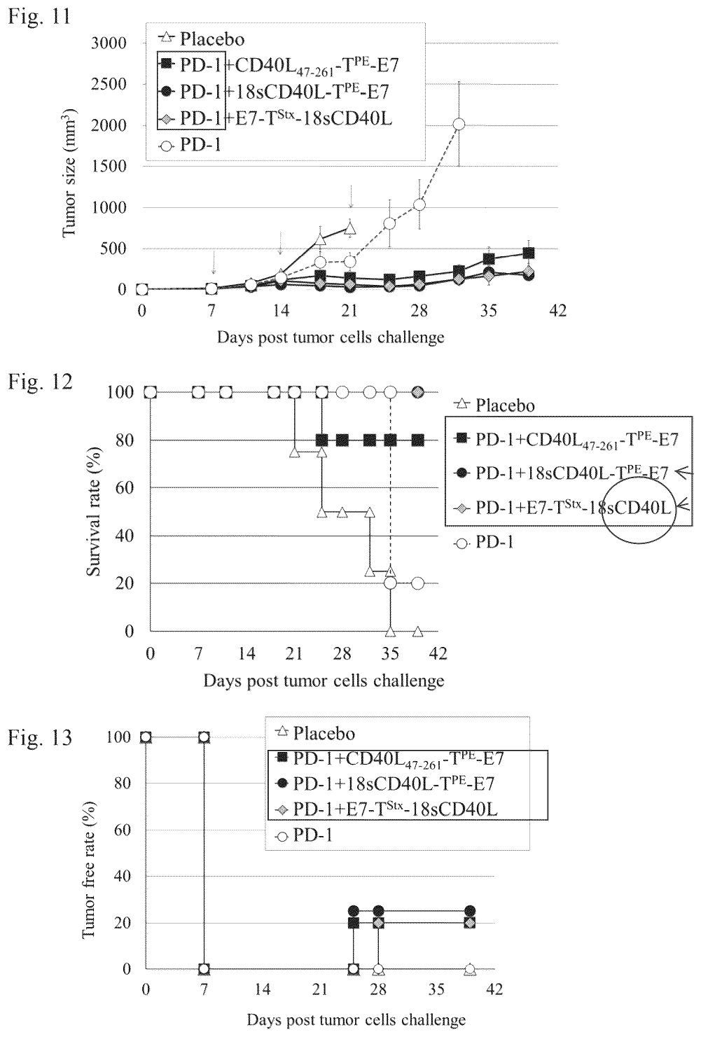

The inoculated tumor developed rapidly in the placebo group, in which two animals died on day 25 and thus the data for the placebo group were shown only until day 21 (FIG. 11). The tumor masses in the animal groups receiving anti-PD-1 antibody in combination with CD40L₄₇₋₂₆₁-T^{PE}-E7, 18sCD40L-T^{PE}-E7, or E7-T^{Stk}-18sCD40L were almost completely suppressed at least during the entire experimental period (last day is Day 39). The tumors in the animal group receiving anti-PD-1 antibody only were initially well controlled, however, rapidly grew after ceasing immunization. The results indicated that the combinations of the invention can effectively suppress tumor growth.

The animal groups receiving anti-PD-1 antibody in combination with 18sCD40L-T^{PE}-E7, and E7-T^{Stk}-18sCD40L, respectively, remained 100% survival rate on day 39, and the group receiving anti-PD-1 antibody in combination with CD40L₄₇₋₂₆₁-T^{PE}-E7 kept at 80% survival rate on day 39, while in the placebo group all mice died on day 35, and the group receiving PD-1 only declined to 20% survival rate on day 35 (FIG. 12). The results indicated that the combinations of the invention could effectively maintain the survival rate in the animal tumor model.

No tumor-free animals could be found in the placebo and PD-1 only groups during the entire experimental period (day 39) (FIG. 13). Notably, in the animal groups receiving combinations of the invention one animal was found surviving without measurable or palpable tumors. In those tumor-free mice the tumor masses were all eliminated soon after completion of three times of immunizations with the combinations of the invention. Thus, the combinations of the invention could potently suppress tumor growth and exhibit outstanding therapeutic efficacy.

E7-T^{Stx}-18sCD40L Fusion Protein in Combination with an Immune Checkpoint Stimulator in a Mouse Model of HPV Infection

E7-T^{Stx}-18sCD40L fusion protein in combination with an immune checkpoint stimulator antibody (an anti-CD137 antibody) was tested in a mouse HPV 16 tumor model, which was established as described above.

The animal grouping and immunization schedule were shown in Table 2. Female C57BL/6NCrlBltw mice (5 to 6-week-old) were randomly divided into 4 groups (n=5 each group): (1) Group A (placebo; PBS); (2) Group B (E7-T^{Stx}-18sCD40L); (3) Group C (anti-CD137 antibody; catalog no. BE0239, Bio X Cell, Inc.); and (4) Group D (E7-T^{Stx}-18sCD40L in combination with anti-CD137 antibody).

To challenge mice, the HPV tumor cells having a higher concentration of 1×10⁶ in 0.1 mL were inoculated subcutaneously into the left flank of each mouse on day 0. In placebo group, PBS was given on Day 14 and 21. For each vaccination (Table 2), the fusion protein E7-T^{Stx}-18sCD40L was dissolved in PBS, adjuvanted with CpG1826 (50 μg/dose) and then given subcutaneously. The anti-CD137 antibody was given intraperitoneally. All mice were sacrificed on day 41.

TABLE 2

| | E7-T^{Stx}- | CD137 | | | | | | | | |
|---|---|---|---|---|---|---|---|---|---|---|
| | 18sCD40L | mAb | Day | | | | | | | |
| Group | (μg/dose) | (μg/dose) | 0 | 14 | 17 | 21 | 28 | 31 | 35 | 41 |
| A | 0 | 0 | — | — | — | — | — | — | — | Sacrifice |
| B | 50 | 0 | — | X | — | X | — | — | — | |
| C | 0 | 50 | — | Y | Y | Y | — | — | — | |
| D | 50 | 50 | — | X | — | X | Y | Y | Y | |

(Note:
“X”: E7-T^{Stx}-18sCD40L; “Y”: anti-CD137 mAb; “—”: not vaccinated)

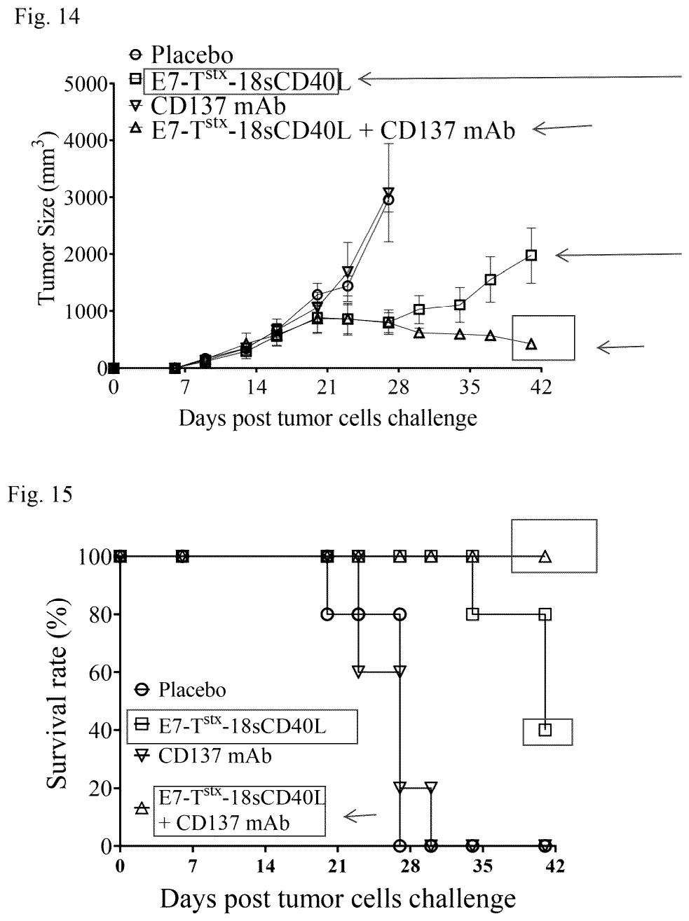

The tumor developed rapidly in the placebo and anti-CD137 antibody only groups, all the mice died around Day 30, which caused no more data could be recorded any further. Surprisingly, the anti-CD137 antibody alone was found making no contribution to tumor suppression here (FIG. 14).

The tumor mass in the group receiving E7-T^{Stx}-18sCD40L only was well controlled initially, started to grow slowly 7 days after the 2^{nd} immunization. Notably, the tumor suppression effect of the fusion protein was maintained for at least one week after the last immunization even though only two vaccinations were administered and a higher amount of tumor cells were inoculated (ten times of the amount used in the aforementioned PD-1 combination study).

The tumor mass in the group receiving E7-T^{Stk}-18sCD40L in combination with anti-CD137 antibody was suppressed and found continuous tumor shrinkage until the end of the experiment on day 42. It could be reasonably speculated that the tumor mass might be able to be cleared completely given a longer observation period. Unexpectedly, the anti-CD137 antibody showed a synergistic effect to the fusion protein E7-T^{Stx}-18sCD40L. The results indicated that the combination of the invention could effectively suppress tumor growth.

The group receiving combination of E7-T^{Stk}-18sCD40L with anti-CD137 antibody remained 100% survival rate until the last observation day, while the group receiving E7-T^{Stx}-18sCD40L declined to 40% survival rate, and both placebo and anti-CD137 antibody only groups declined to 0% survival rate (FIG. 15). The results indicated that the combination of the invention could effectively maintain the survival rate.

Accordingly, a combination of an E7-carrying fusion protein with an immune checkpoint antibody capable of activating T cell, such as an anti-PD-1 antagonist antibody, or an anti-CD137 agonist antibody, could exhibit an excellent tumor suppression effect and be used for treating tumors or abnormal cell proliferation.

### Example 5

Efficacy Analyses: HBx-preS1-T^{Stx}-18sCD40L Fusion Protein in Combination with an Immune Checkpoint Antibody in a Mouse Model of HBV Infection

A combination of HBx-preS1-T^{Stx}-18sCD40L with an immune checkpoint inhibitor (an anti-PD-1 antibody) or an immune checkpoint stimulator (an anti-CD137 antibody) is tested for the efficacy in a HBV-infection mouse model.

Mice are divided into several groups: control (PBS), vaccine (HBx-preS1-T^{Stx}-18sCD40L), PD-1 (anti-PD-1 antibody), CD137 (anti-CD137 antibody), PD-1 combo (anti-PD-1 antibody and HBx-preS1-T^{Stx}-18sCD40L), and CD137 combo groups (anti-CD137 antibody and HBx-preS1-T^{Stx}-18sCD40). The fusion protein is adjuvanted with CpG1826 and given subcutaneously. Antibodies were given intraperitoneally.

A HBV-infection mouse model is an AAV-HBV mouse model described in U.S. Pat. No. 10,058,606 B2 and can be suitably modified if needed. Briefly, male mice (5-6 weeks old) are used to establish an animal model carrying a long-term hepatitis B virus. A mixture of pAAV/HBV1.2 plasmid and saline is intravenously injected into the mouse tail vein at a high pressure (hydrodynamic injection, HDI) in a fast mode to force the plasmid to penetrate the cell membrane and enter liver cells. The plasmid-carrying liver cells express hepatitis B virus proteins. Virus will assemble inside the liver cells and release into blood. This animal model emulates a human patient afflicted with chronic hepatitis B symptoms.

Twenty-eight days after the high-pressure injection, mice in each group receive three doses of fusion proteins and/or indicated antibodies on Days 0, 7, 14 with a 7 day interval. The body weights are measured on the same day of high-pressure injection, and on Day 0, 7, 14, 21, 32, and 42. Blood is collected for analyses of alanine aminotransferase (ALT), bilirubin, viral DNA, and surface antigen (HBsAg). Mice are sacrificed 82 days after the first vaccination, and liver core antigen (HBcAg) quantity is analyzed.

The administration of HBx-preS1-T^{Stx}-18sCD40 in combination with an anti-PD-1 antibody or an anti-CD137 antibody is expected to exhibit an outstanding therapeutic effect on HBV infection. For example, said combination is expected to reduce viral DNA load, HBsAg and HBcAg, and induce an HBx-specific immune response. Use of said combination (i.e., the HBx-carrying fusion protein combined with an immune checkpoint modulator) is expected to effectively inhibit the proliferation of hepatitis B virus in liver cells and suppress hepatitis B virus infection in HBV patients.

In summary, the novel antigen-carrying fusion proteins can elicit a potent antigen-specific T cell immune response due to its unique protein design and mechanism of action. An immune checkpoint modulator is able to restore T cell function by antagonizing an inhibitory immune checkpoint (e.g., PD-1, PD-L1, PD-L2, CTLA-4, LAG3, TIGIT, CD96, CD122R, TIM3, VISTA, CEACAMI, SIGLEC-7, SIGLEC-9, SIGLEC-15, KIRi, CD200R, BTLA, and ILT2) or activate T cell by agonizing a stimulatory immune checkpoint (e.g., CD137, OX40, GITR, ICOS, CD27, CD28, CD40, 5 KIRs, CD226 and CD244). When at least one suitable antigen is applied in the fusion protein and a suitable immune checkpoint modulator is selected, both of which contribute to upgrade T cell immunity and could achieve a great therapeutic efficacy when they are used in combination. Therefore, the invention provides an original inventive conception of using the combinations that are not disclosed in any prior publications for treating tumors and infectious diseases.

All references cited and discussed in this specification are incorporated herein by reference in their entireties and to the same extent as if each reference was individually incorporated by reference.

1. A combination, comprising:
(a) an immune checkpoint antibody capable of activating a T cell; and
(b) a fusion protein, comprising:
(i) a CD40-binding domain, which is a CD40 ligand (CD40L) or a functional fragment thereof comprising an amino acid sequence that is at least 95% identical to SEQ ID NO: 19, said CD40L or functional fragment thereof consisting of 154-261 amino acid residues in length;
(ii) an antigen, which is a tumor antigen or an antigen of a pathogen;
(iii) a translocation domain, located between the CD40-binding domain and the antigen, said translocation domain being selected from the group consisting of:
(iii-1) a Shiga toxin (Stx) translocation peptide; and
(iii-2) a Pseudomonas Exotoxin A (PE) translocation peptide; and
(iv) a furin and/or cathepsin L cleavage site, located between the CD40-binding domain and the translocation domain,
wherein when the translocation domain is the Stx translocation peptide, the antigen is located at the N-terminal of the fusion protein; and when the translocation domain is the PE translocation peptide, the CD40-binding domain is a CD40L monomer and located at the N-terminal of the fusion protein.

2. The combination of claim 1, wherein the translocation domain is the PE translocation peptide, the PE translocation peptide consisting of 26-112 amino acid residues in length and comprising an amino acid sequence that is at least 95% identical to SEO ID NO: 5, 6, 7, 8, or 9.

3. The combination of claim 1, wherein the translocation domain is the PE translocation peptide, the PE translocation peptide consisting of 26-112 amino acid residues in length and comprising an amino acid sequence of SEQ ID NO: 5.

4. The combination of claim 1, wherein the translocation domain is the Stx translocation peptide, the Stx translocation peptide consisting of 8-84 amino acid residues in length and comprising an amino acid sequence that is at least 95% identical to the amino acid sequence selected from the group consisting of SEQ ID NOs: 12, 13, 14, 15 and 16.

5. The combination of claim 1, wherein the translocation domain is the Stx translocation peptide consisting of 8-84 amino acid residues in length and comprises an amino acid sequence of SEQ ID NO: 12.

6. The combination of claim 1, wherein the furin and/or cathepsin L cleavage site permits removal of the CD40-binding domain away from the fusion protein via furin and/or cathepsin L cleavage.

7. The combination of claim 1, wherein the furin and/or cathepsin L cleavage site comprises an amino acid sequence of SEQ ID NO: 1 or 2.

8. The combination of claim 1, further comprising a peptide linker, wherein the furin and/or cathepsin L cleavage site is present in said peptide linker.

9. (canceled)

10. The combination of claim 1, wherein the CD40 ligand (CD40L) or the functional fragment thereof comprises the amino acid sequence of SEQ ID NO: 19 with 154-261 amino acid residues in length.

11. The combination of claim 1, wherein the antigen is a tumor antigen, said tumor being selected from the group consisting of breast cancer, colon cancer, rectal cancer, bladder cancer, endometrial cancer, kidney cancer, gastric cancer, glioblastoma, hepatocellular carcinoma, bile duct cancer (cholangiocarcinoma), small cell lung cancer, non-small cell lung cancer (NSCLC), melanoma, ovarian cancer, cervical cancer, pancreatic cancer, prostate cancer, acute myelogenous leukemia (AML), chronic myelogenous leukemia (CML), non-Hodgkin's lymphoma, and thyroid cancer.

12. The combination of claim 1, wherein the antigen is an antigen of a pathogen, said pathogen being selected from the group consisting of Human Papillomavirus (HPV), Human Immunodeficiency Virus-1 (HIV-1), Influenza Virus, Dengue Virus, Hepatitis A Virus (HAV), Hepatitis B Virus (HBV), Hepatitis C Virus (HCV), Hepatitis D Virus (HDV), Hepatitis E Virus (HEV), Severe Acute Respiratory Syndrome-Associated Coronavirus (SARS-COV), Severe Acute Respiratory Syndrome Coronavirus 2 (SARS-CoV2), Middle East Respiratory Syndrome Coronavirus (MERS-Cov), Epstein-Barr Virus (EBV), Zika Virus, Rabies Virus, Variola Virus, Chikungunya Virus, West Nile Virus, Poliovirus, Measles Virus, Rubella Virus, Hantavirus, Japanese Encephalitis Virus, Coxsackievirus, Echovirus, Enterovirus, Mumps Virus, Varicella-Zoster Virus (VZV), Cercopithecine Herpesvirus-1 (CHV-1), Yellow Fever Virus (YFV), Rift Valley Fever Virus, Lassa Virus, Marburg Virus, Ebolavirus, Norovirus, Rotavirus, Adenovirus, Sapovirus, Astrovirus, Porcine Reproductive and Respiratory Syndrome Virus (PRRSV), African Swine Fever Virus (ASFV), Classical Swine Fever Virus (CSFV), Porcine Circovirus 2 (PCV2), Foot-and-Mouth Disease Virus (FMDV), Porcine Epidemic Diarrhea Virus (PEDV), Swine Vesicular Disease Virus (SVDV), Pseudorabies Virus (PRV), Transmissible Gastroenteritis Virus (TGEV), Newcastle Disease Virus (NDV), Infectious Bronchitis Virus (IBV), Infectious Bursal Disease Virus (IBDV), Mycoplasma hyopneumoniae, Rickettsia prowazekii, Rickettsia typhi, Orientia tsutsugamushi, Borrelia burgdorferi, Yersinia pestis, Plasmodium vivax, Plasmodium malariae, Plasmodium falciparum, Plasmodium ovale, Bacillus anthracis, Clostridium Difficile, Clostridium Botulinum, Corynebacterium diphtheriae, Salmonella enterica serovar Typhi, Salmonella enterica serovar Paratyphi A. Shiga toxin-producing E. coli (STEC), Shigella dysenteriae, Shigella flexneri, Shigella boydii, Shigella sonnei, Entamoeba histolytica, Vibrio cholerae, Mycobacterium tuberculosis, Neisseria meningitidis, Bordetella pertusis, Haemophilus influenzae type B (HiB), Clostridium tetani, Listeria monocytogenes and Streptococcus pneumoniae.

13. The combination of claim 1, wherein the immune checkpoint antibody is an antagonist antibody capable of targeting an inhibitory immune checkpoint, an agonist antibody capable of targeting a stimulatory immune checkpoint, or a bispecific antibody capable of targeting two immune checkpoints.

14. The combination of claim 13, wherein said inhibitory immune checkpoint is selected from the group consisting of PD-1, PD-L1, PD-L₂, CTLA-4, LAG3, TIGIT, CD96, CD122R, TIM3, VISTA, CEACAMI, SIGLEC-7, SIGLEC-9, SIGLEC-15, KIRI, CD200R, BTLA, and ILT2.

15. The combination of claim 13, wherein said stimulatory immune checkpoint is selected from the group consisting of CD137, OX40, GITR, ICOS, CD27, CD28, CD40, KIRS, CD226 and CD244.

16. The combination of claim 2, wherein the fusion protein further comprises a CD28-activating peptide located between the CD40-binding domain and the form and/or cathepsin L cleavage site, the CD28-activating peptide consisting of 28-53 amino acid residues in length and comprising an amino acid sequence selected from the group consisting of SEQ ID NO: 35, 36 and 37.

17. The combination of claim 2, wherein the PE translocation peptide comprises the amino acid sequence selected from the group consisting of SEQ ID NOs: 5, 6, 7, 8 and 9.

18. The combination of claim 2, wherein the Stx translocation peptide comprises an amino acid sequence selected from the group consisting of SEQ ID NOs: 12, 13, 14, 15 and 16.

19. A combination, comprising:
(a) an immune checkpoint antibody capable of activating a T cell; and
(b) a fusion protein, comprising:
(i) a CD40-binding domain, which is a CD40 ligand (CD40L) or a functional fragment thereof comprising an amino acid sequence that is at least 90% identical to SEQ ID NO: 19, said CD40L or functional fragment thereof consists of 154-261 amino acid residues in length;
(ii) an antigen, which is a tumor antigen or an antigen of a pathogen;
(iii) a translocation domain, located between the CD40-binding domain and the antigen, said translocation domain being selected from the group consisting of:
(iii-1) a Shiga toxin (Stx) translocation peptide, comprising an amino acid sequence of SEQ ID NO: 12; and
(iii-2) a Pseudomonas Exotoxin A (PE) translocation peptide, comprising an amino acid sequence of SEQ ID NO: S; and
(iv) a furin and/or cathepsin L cleavage site, located between the CD40-binding domain and the translocation domain:
wherein when the translocation domain is the Stx translocation peptide, the antigen is located at the N-terminal of the fusion protein; and when the translocation domain is the PE translocation peptide, the CD40-binding domain is a CD40L monomer and located at the N-terminal of the fusion protein.

20. A method for eliciting an antigen-specific cell-mediated immune response, comprising: administering a therapeutically effective amount of the combination of claim 1 to a subject in need thereof, and thereby eliciting an antigen-specific cell-mediated immune response in the subject in need thereof.

21. A method for treating a tumor in a subject in need thereof, comprising: administering to the subject in need thereof a therapeutically effective amount of the combination of claim 1, wherein the antigen of the fusion protein is a tumor antigen, and thereby treating the subject in need thereof.
